(19) United States
(12) Patent Application Publication
(10) Pub. No.: US 2022/0354783 A1
(43) Pub. Date: Nov. 10, 2022

(54) INJECTABLE PROLONGED-ACTION COMPOSITIONS FOR USE IN THE TREATMENT OF NAIL DISEASE AND/OR FOR PROMOTING NAIL GROWTH

(71) Applicant: EDIX-O SARL, LUXEMBOURG (LU)

(72) Inventors: Roland CHERIF-CHEIKH, CASTELLDEFELS-BARCELONA (ES); Frederic LACOMBE, SANT CUGAT DEL VALLES (ES); Laurence LACHAMP, GAVA BARCELONA (ES); Xavier Julia CAMPRODON, BARCELONA (ES); Albert DOMENECH AGUERA, BARCELONA (ES); Marta SERRANO CARRENO, BARCELONA (ES)

(73) Assignee: EDIX-O SARL, LUXEMBOURG (LU)

(21) Appl. No.: 17/264,937

(22) PCT Filed: Jul. 31, 2019
(86) PCT No.: PCT/EP2019/070646

§ 371 Date: Feb. 1, 2021

(30) Foreign Application Priority Data
Aug. 1, 2018 (EP) 18306051.6
Apr. 18, 2019 (EP) 19305513.4

(51) Int. Cl. A61K 9/00; A61K 31/137; A61K 31/4412; A61K 31/5375; A61K 31/496; A61P 31/10; A61K 31/573; A61K 31/4174; A61K 8/97; A61K 8/49; A61K 8/73; A61K 47/34; A61Q 3/02; A61N 5/06; A61Q 17/00

(52) U.S. Cl. CPC A61K 9/0024; A61N 2005/067; A61K 31/4412; A61K 31/5375; A61K 31/496; A61P 31/10; A61K 31/573; A61K 31/4174; A61K 8/97; A61K 8/4953; A61K 8/736; A61K 47/34; A61Q 3/02; A61K 9/0053; A61N 5/0624; A61Q 17/005; A61K 31/137

(57) ABSTRACT

Injectable prolonged-action compositions for use in the treatment of nail diseases, including potentially promoting the growth of the nail, or for use promoting the growth of the nail. The compositions are administered subcutaneously, and optionally as an implant. The compositions may be in solid or non-solid form and include one or more solid biodegradable sustained-release polymers, and optionally one or more active substances.

The invention relates to injectable prolonged-action compositions for use in the treatment of nail diseases, including potentially promoting the growth of the nail, or for use promoting the growth of the nail.

### STATE OF THE ART

Among the various diseases that can affect the nails or extremities are inflammatory diseases such as psoriasis, or lichen planus; nail tumors such as glomus tumors, myxomatous tumors, myxoid skin cysts; infections such as paronychia, panaris, mycosis and other fungal conditions such as onychomycosis also known as nail fungus or nail, onychatrophy, leukonychia, onychogryphosis, koilonychia, nail melanonychia, subungal hyperkeratosis, onychorrhexia, paronychia, diseases of chemical, infectious, inflammatory or other traumatic origin such as alopecia, Darrier's disease and other common diseases known to the person skilled in the art, not limited to fungal mycoses.

Mycoses and other fungal infections can also affect the skin, mucous membranes and hair.

Onychomycosis is the most common nail condition, affecting 6-9% of the general population. It is rare in children and its frequency increases with age, affecting 50% of people over 70. Due to their often reduced mobility and their polypharmacy, these people are not adequately treated today.

In the United States alone, prevalence is estimated at over 35 million people.

Globally, 15% of the general population will suffer from at least one nail fungus in their lifetime.

There are many factors that increase the propensity to suffer from onychomycosis: a possible genetic predisposition, microtrauma, associated diseases (psoriasis, diabetes, poor blood circulation in the feet, immunodeficiency, etc.), and the presence of a number of other factors that can increase the risk of developing onychomycosis), certain drug treatments (immunosuppressants, chemotherapy, corticoids, etc.), age-related factors, and above all environmental factors (heat and humidity, heavy sweating, continuous wearing of closed shoes, walking barefoot on public ground, among others).

Onychomycosis is a fungal infection of the nail bed, matrix or nail tablet. The first clinical feature of onychomycosis is distal onycholysis (separation of the nail plate from the nail bed), subungual hyperkeratosis and a discoloured dystrophic nail.

Patients with onychomycosis are usually embarrassed by the imperfections of their nail(s) and one of the recognised major impacts of the condition is the social impact as well as the loss of self-confidence.

The symptoms of the disease are initially only aesthetic with the appearance of spots on the nail, but if the disease is not treated, the infection can spread and be accompanied by physical symptoms, sometimes limiting mobility with difficulty walking (or even loss of dexterity) and may be associated with pain. The disease can also indirectly decrease peripheral circulation and lead to venous stasis, which can aggravate medical conditions such as ulcers in diabetic patients.

Fungal nail infections can also spread to other parts of the body and potentially to other people.

In the case of nail fungus or onychomycosis, different species of pathogenic fungi can be identified. The most frequent and in more than 70% of cases are Dermatophytes, in particular Trichophyton rubrum, Trichophyton interdigital or Trichophyton mentagrophytes.

The remaining 30% are yeasts of the Candida type in general Candida parapsilosis, Candida guilliermondii, Candida Albicans, or non-dermatophytic moulds such as Aspergillus spp, Scopulariosis spp, Brevicaulis spp, Fusarium spp, Acremonium spp and Neoscytalidium spp.

Other common dermatophyte pathogens include Epidermophyton floccosum, Trichophyton violaceum, Microsporum gypseum, Trichophyton tonsurans Tricophyton schoenleinii or Trichophyton souadenense.

There are five subtypes of onychomycosis identified and associated with the degree of severity.

Distal subungual onychomycosis (DLSO), however, represents the most common cases even though patients tend to consult their doctor late when the onychomycosis progresses closer to the nail base area endangering the matrix itself. Until then, they rarely associate their changes in nail morphology with nail disease.

Even if the most common pathologies are onychomycoses, the object of the invention also makes it possible to treat all mycoses as well as diseases of the nail (onychopathies) such as those indicated above and in particular psoriasis or panaris, as well as to improve and restore alterations in the morphology of the nail (onychodystrophies), whether or not related to the above-mentioned pathologies. The object of the invention can also treat in a general way the so-called extremity diseases known to person skilled in the art.

In the case of these infections, the most commonly used current drug treatments consist of the daily administration over several months, or even a whole year, of topical pharmaceutical forms and/or oral forms for the eradication of fungi in the case of dermatomycoses and the aesthetic recovery of the affected areas.

It is probable and often proven that in order for the treatment to be definitive or to avoid any recurrence, it is necessary to treat for more than a year. However, this is not possible in practice with the current application or daily intake treatments.

Antifungals used to treat nail fungus can preferably include Nystatin, Amphotericin B, Abafungin, Benzalkonium chloride, Caspofungin, Cetrimide, Clioquinol, Copper sulphate, Haloprogin, Echinocandins, Flucytosine, Mycobactovir, Novexatin, Natamycin, Cetylpyridium chloride, Benzylamine butenafine, Benzoxaboroles (cyclic derivatives of boronic acids), Albaconazole, Arasertaconazole, Bifonazole, Butoconazole, Clotrimazole, Eberconazole, Econazole, Eficonazole, Fenticonazole, Fluconazole, Fosravuconazole, Isoconazole, Irtemazole, Isavuconazole, Ketoconazole, Liarozole, Lianozole, Luliconazole, Miconazole, Oxadiazole, Oxiconazole, Posaconazole, Pramiconazole, Ravuconazole, Sertaconazole, Sulconazole, Terconazole, Thiazole, Thiabendazole, Thiadiazole, Thiamazole, Piroctone olamine, Climbazole, Tioconazole, Voriconazole, Amiodarone, Naftifine, Tavaborole, Butenafine, Flucytosine, Griseofulvin, Caspofungin, Micafungin, Nitric Oxide, Sodium Oxychlorosene, Povidoneiodine, Thiocarbonate tolnafate, Sulbentine, Zync Pyrithione, and more particularly among Terbinafine, Ciclopirox, Ciclopiroxolamine, Itraconazole, and Amorolfine.

Oral treatments may contain, for example, ketoconazole, fluconazole, isavuconazole, itraconazole, posaconazole, or voriconazole; echinocandins such as anidulafungin, caspofungin, micafungin, flucytosin, griseofulvin, and terbinafine; amphotericin B and in particular ketoconazole, itraconazole, terbinafine hydrochloride, fluconazole, griseofulvin, terbinafine and in case of hospital emergency treatment posaconazole or posaconazol and voriconazole. These antifungals may also be used for the implementation of the present invention. However, these oral antifungals are associated with mild systemic effects (migraines, skin rashes, loss of taste or photosensitivity), or serious effects such as heart or liver problems which severely limit their use, particularly in the most affected patient populations.

Topical therapies with antifungal agents such as fluconazole, ketoconazole, miconazole, terbinafine, tolnaftate, sertaconazole, eberconazole, fenticonazole, oxiconzale, clotrimazole, bifonazole, are an alternative for patients in whom oral antifungal therapy is contraindicated.

All these antifungals can also be used for the implementation of the present invention.

For the treatment of local cutaneous mycoses which are theoretically easy to access, but difficult to apply for most patients due, for example, to the limitation of their physical ability to reach the area to be treated. Medicinal forms such as creams, gels, ointments, sprays, powders, aerosols and lotions are used. Varnishes containing one or more antifungals are also a frequently used treatment, although they have many side effects, such as irritation, itching, rashes, inflammation, contact dermatitis, burns to the skin near the area of administration, as well as nail damage (discoloured nails, fragile nails or brittle nails).

These medicinal forms of topical application to the infected area do not allow direct access to all the fungi often distributed in the various keratin layers of the nail and not only on its surface, and thanks to the fungistatic and fungicidal effect of the therapeutic agents incorporated in the preparations, when the results are satisfactory (less than 20% of cases), the eradication of the fungus and the restoration of the healthy morphological aspect of the nail are obtained after periods of treatment that can last from a few weeks to several months.

Nail fungus, due to its difficult access, is still one of the most difficult to treat today. Indeed, fungi can colonise the nail from its bed, which is impossible to treat with a topical product, and gradually spread, affecting the keratinised structure of the nail. If the infection is not stopped in time, it can go as far as colonising the entire nail down to the matrix, with serious systemic consequences for patients, for example for diabetic patients. Furthermore, only 5% of the onychomycosis population in the United States of America and 8% of the European population treats onychomycosis. The vast majority of the affected population does not try to resolve this infection, which can lead to late treatment and further complicates the difficulty of treatment.

The case of advanced mycosis where the matrix is infected or threatened with infection by a progression of the disease, which is one of the first focuses of the invention, generally often involves the use of oral treatments, as topical treatments and other treatments of the LASER type do not penetrate well to the base of the infection, and therefore cannot cure at this specific point of nail renewal.

As a result, at the nail level, it is difficult to access the fungus using conventional topical treatments. For this reason, systems have been developed to promote direct treatment of the nail and facilitate the passage of therapeutic agents through the nail.

One of the therapeutic solutions being considered are therapeutic varnishes described for example in the international application WO 2010/086723. They contain film-forming agents and solvents that evaporate after application leaving the active substance deposited on the nail.

For example, international application WO 2007/147052 describes a composition including terbinafine, for use in the treatment of onychomycosis by topical application. The composition consists of a delivery vehicle comprising a non-polymeric crystallisation inhibitor, a film-forming agent and a volatile solvent.

International application WO 2011/79234 describes topical, antifungal compositions comprising an antifungal agent, a zwitterionic surfactant or its content derivative, a carboxylic acid, a low molecular weight alcohol, and water.

Another example is Valeant Pharmaceuticals' Penlac® varnish (8% ciclopirox solution), which has been approved by the Food and Drug Administration in the United States for the topical treatment of mild to moderate onychomycosis.

These varnishes must be applied very regularly and generally contain a maximum of 10% therapeutic agent (except for Pfizer's Trosyd® product, which can contain up to 28% tioconazole). These drugs offer a local surface treatment for nail fungus, but do not attack the nest of the infection or create an active barrier or brake to its spread. In addition, permeation through the nail is very low and only a tiny part of the dose reaches the area to be treated directly.

It is also important to consider the environmental impact and the use of large quantities of organic solvents. More recently, in order to compensate for this disadvantage, new film-forming compositions have been developed that reduce the quantities of organic solvents; however, they do not change the dosage or duration of treatment.

Substantial research efforts have been focused on the development of systems that increase the transungual permeation of therapeutic agents.

More recently, based on transdermal systems, formulations that can be administered by iontophoresis have been developed, as for example described in application WO2008/121709.

To maximise the contact of the active substance with the infection, there are also proposals involving piercing the nail in multiple places using a LASER beam and a deposit of formulations containing the therapeutic agents.

Such processes are described in international patent application WO 02/11764 and more recently in application WO2011/071137. These highly invasive and painful approaches are also expensive. They offer the advantage of targeting the infection under the nail but do not, for example, address possible collateral infections due to the contagious nature of fungal infections.

Onychomycosis is usually also associated with fungal infections of adjacent areas such as fingers, toes, feet and hands. Furthermore, this type of administration, due to the size of the holes made in the nail, only allows the supply of an infinitesimal quantity of active substance that does not present a delaying effect that can treat the nail over several weeks or months without renewing the product.

At the matrix level, it is not possible to envisage a satisfactory topical treatment that acts effectively deep into the living tissue. When infection is present at this level, the matrix will continue to produce a diseased, infected nail. In these advanced cases of fungal infections, despite their side effects, oral medications should be considered, which may be preferable to combine with topical forms.

As mentioned above, due to limitations arising from lack of accessibility, effectiveness, frequency and duration of treatment, doctors currently have no other option than to resort to systemic oral treatment alternatives.

These oral tablet forms are usually administered in high doses daily for periods limited to 3 months, alone or in combination with topical forms containing antifungal agents applied daily and require regular patient monitoring.

These oral treatments involve high exposure of the whole body for a long period of time and have the disadvantage of causing significant side effects, particularly liver toxicity, limiting their routine use in the most advanced cases of the disease.

In all cases, and whatever the treatments, one must wait until complete regrowth of the nail (6 months for the hand and 12 months for the toes) to be able to obtain a total remission; this imposes an extreme discipline of daily follow-up throughout the entire duration of the treatments. By remission is meant the absence of infection and the return to a normal aesthetic appearance, a necessary criterion of remission according to the person skilled in the art.

For example, today there are no easy-to-use medicines to treat onychomycosis or nail diseases. Current treatments generally do not allow a cure to be achieved, as it depends on strict adherence to daily treatment over a year or on the limit of treatments with the risk of side effects, and also often on the patient's ability to take them.

Patients often stop their treatment on their own as soon as the disabling aesthetic aspect seems to have disappeared or has diminished. In such cases, there often remain colonies of fungus which then reproduce rapidly and lead to a relapse and recurrence of the infection (absence of mycological cure).

As indicated above, one of the reasons for the failure of current treatments is the lack of observance or compliance, i.e. the patient's failure to follow his doctor's prescriptions when taking these drug treatments and the associated hygienic and dietary rules. The treatments are restrictive and sometimes complex. For example, it is very complicated for elderly patients with reduced mobility to use daily topical forms that are difficult to apply to infected toenails requiring special physical fitness.

Adherence is a key element in the success of a drug therapy, especially in those treatments where it is important to have antifungal products permanently present at the infected sites.

This non-observance is recognised as one of the main causes of the failure of these treatments.

Another reason for these failures is related to the time limits for taking oral forms, which alone are capable of ensuring the absence of infection at the birth of the nail at the level of the womb. Furthermore, oral forms are the only systemic treatments for dermatophytosis or other skin, hair or nail infections. In addition, systemic antifungals are used when the germ in question—a microscopic fungus—is resistant or when local treatment is impossible. However, these treatments are only prescribed in extreme cases and are not indicated for the general population.

In emergency hospital treatment and in the case of serious fungal infections such as Aspergillosis, or mucocutaneous candidiasis and in fungal infections in immunocompromised patients, the nursing staff have access to injectable, intravenous antifungal treatments. For example, there are intravenous solutions of amphotericin B, fluconazole, voriconazole, itraconazole or posaconazole.

There are already known treatments with injections at the fingertips and around the nail. The treatment of psoriasis can, for example, lead to injections of anti-inflammatory glucocorticoids into the lesions. For example, the administration of triamcinolone in the proximal nail fold, along the lateral fold and on either side, in the matrix or in the nail bed involves local anaesthesia as these areas are known to be very sensitive. This type of treatment often leads to side effects such as nail atrophy or subungual haemorrhages.

Patent applications have been filed for the treatment of nail diseases and in particular onychomycosis using biodegradable drug delivery systems formulated for implantation in the nail and its surrounding tissues for the treatment of various nail conditions.

Such a system is described in application WO2006/086888 (equivalent to american application US2008/0299165).

The systems include compositions that can also be formulated as solids, especially in the case of the subsequent international applications WO 2007/139804 and WO 2011/086723.

However, the implantation of a solidifiable or solid formulation under the nail or in the surrounding tissues in case of disease and especially in case of onychomycosis has many disadvantages and can be very painful.

Indeed, there is not enough space in these specific areas to administer a composition with sufficient volume for a treatment over several months. Nor is it desirable to perform an invasive injection procedure in or near the sick or infected area which will add trauma or even encourage the spread of infection.

These are areas that are particularly innervated and vascularised or too close to a joint, in which the insertion of a needle and the administration of a solid or solidified form pose problems of trauma and scarring or can even trigger other pathologies linked for example to osteoarthritis, which are not favourable to the healing of the infected tissue, which is the objective of the treatment.

It is also not possible to multiply the number of solidifiable or solid forms injected in these areas or to easily repeat these injections.

Indeed, in the presence of combined pathologies, different active substances and in particular different antifungals are often necessary for the treatment of these pathologies and the limited space under the nail or in the surrounding tissues does not allow the injection of these treatments combining several active substances in sufficient quantities.

Indeed, it is now recognised that one of the additional reasons for the failure of treatments for nail diseases is that they are often caused by different types of pathogens and often have associated consequences such as inflammation or other diseases.

As an open-ended example, some fungi are sometimes resistant to terbinafine and others to ciclopirox. However, as indicated above, there is currently no means for the patient to treat these fungi simultaneously and specifically without exposing the organism to high doses of active therapeutic ingredients with a certain toxicity.

Another disadvantage of the solutions proposed in these patent applications is that they do not target the upstream area of the nail matrix, which is a priority objective of in-house treatments, whether injectable or oral.

This upstream area is also not targeted for external treatments such as topicals or varnishes.

For all the reasons mentioned, today a large part of the population suffering from nail diseases has no treatment at all.

To sum up, it appears that the therapeutic problems posed by the treatment of nail diseases and in particular onychomycosis, such as lack of compliance, lack of efficacy, duration and frequency of treatment have not found satisfactory solutions to date.

### SUMMARY OF THE INVENTION

The present invention therefore has as subject matter a composition for use in the treatment of nail diseases and/or for promoting nail growth, characterized in that it comprises one or more solid biodegradable sustained-release polymers, and optionally one or more active substances and in that it is in the form of a device intended to be administered subcutaneously at a distance greater than 1 cm from the edge of the nail on the side of its proximal end preferably in the finger with the exception of the last phalanx and also outside a joint area, preferably in the penultimate phalanx remote from the tendon and its bony insertion areas.

The present invention has as subject matter in particular a composition for its use in the treatment of nail diseases and/or for promoting nail growth, characterized in that it comprises one or more solid biodegradable sustained-release polymers, and optionally one or more active substances and in that it is in the form of a device intended to be administered subcutaneously in the finger at a distance greater than 1 cm from the edge of the nail on the side of its proximal end preferably except for the last phalanx and also outside a joint area, preferably in the penultimate phalanx remote from the tendon and its bony insertion areas.

The compositions according to the invention described above may be administered subcutaneously at a distance of more than 1 cm from the edge of the nail on the side of its proximal end, preferably in the finger with the exception of the last phalanx.

In a preferred mode, the compositions according to the invention may be administered subcutaneously in the finger at a distance of more than 1 cm from the edge of the nail, preferably towards its proximal end, with the exception of the last phalanx.

### BRIEF DESCRIPTION OF THE FIGURES

FIG. 1: In Vitro Release Graph:

Comparison between the different compositions prepared according to Example 1.

FIG. 2: In Vitro Release Graph:

Comparison between the different compositions prepared according to Example 18.

FIG. 3: In Vitro release graph of the compositions according to Example 2 and Example 6.

FIG. 4: In Vitro release graph of the composition according to Example 16.

FIG. 5: Photo of the halo of an agar plate after implantation of a compound according to Example 1 (1) on seeds of Candidae Parapsilosis strains incubated at 35° C.

FIG. 6: Photo of the halo of an agar plate after implantation of a compound according to Example 2 on seeds of Candidae Parapsilosis strains incubated at 35° C.

FIG. 7: Halo photo of an agar plate after implantation of a compound according to Example 19 on seed of Trichophyton Rubrum strains incubated at 35° C.

FIG. 8: Implant inhibition halo activity curve from Example 1 (3) vs. Trichophyton Rubrum seeds incubated for 75 days at 35° C.

FIG. 9: Implant inhibition halo activity curve according to Example 2 on seeds of Candidae Parapsilosis strains incubated for 168 days at 35° C.

FIG. 10a: Implant inhibition halo activity curve according to Example 19 on seeds of Candidae Parapsilosis strains incubated for 115 days at 27 and 35° C.

FIG. 10b: Implant inhibition halo activity curve according to Example 19 on Trichophyton Rubrum strain seeds incubated for 135 days at 35° C.

FIG. 11: Photo of a composition implant described in Example 18 (3) after 84 days of In Vivo implantation.

FIG. 12: Quantity of active substance released as a function of time from a device according to Example 1 (4)

FIG. 13: Remanence versus time curve of a device according to Example 18 (3)

FIG. 14: Remanence versus time curve of a device according to Example 25

FIG. 15: In Vitro release graph of the composition according to Example 26.

FIG. 16: In Vitro release graph of the composition according to Example 27.

FIG. 17a: Photo of an implant obtained according to Example 1 (4)—side view

FIG. 17b: Photo of an implant obtained according to Example 1 (4)—sectional view

FIG. 18: Photo of an implant obtained according to Example 7—side view

FIG. 19: Photo of an implant obtained according to Example 8—side view

FIG. 20: Photo of an implant obtained following Example 16—side view

FIG. 21: Photo of an implant obtained according to Example 18 (3)—side view

FIG. 22: Photo of an implant obtained following Example 26—side view

FIG. 23: Photo of an implant obtained according to Example 27—profile view.

FIG. 24: Schematic side view of one of the preferred settlement areas

### DETAILED DESCRIPTION OF THE INVENTION

The edge of the nail on the side of its proximal end is understood to mean more particularly the proximal area of the nail on the side of the matrix and the lunula limited by the cuticle.

The affected area or area to be treated is defined as a minimum area, which is defined as one end, for example the nail of a finger or toe, which can extend to more than one end, nail, finger or toe, until it can touch all the patient's extremities, nails, fingers and toes.

Administration or re-administration will preferably be carried out on the dorsal latero-superior surfaces on either side of the extensor tendon and above the vascular-nervous bundles in these areas.

The object of the present invention is therefore a composition for its use in the treatment of nail diseases and/or for promoting nail growth, characterized in that it comprises one or more solid biodegradable sustained-release polymers, and optionally one or more active substances, and in that it is in the form of a device intended to be administered subcutaneously at a distance greater than 1 cm from the edge of the nail on the side of its proximal end, preferably in the finger with the exception of the last phalanx.

The area of administration of the compositions according to the invention is preferably located in the penultimate phalanx, preferably outside a joint area, and preferably the area of administration is away from the tendon and its bone insertion areas.

The solid biocompatible polymers or copolymers that can be used can be selected from Polymers of lactic acid (PLA), Polymers of glycolic acid, Lactic acid-glycolic acid copolymers (PLGA), Polycaprolactones (PCL), Copolymers of lactic acid and caprolactone, Poly-glycolide-co-lactide-co-caprolactone (PLGC), Polyanhydrides, the copolymer between 1,3-bis (carboxyphenoxypropane) (PCPP) with sebatic acid (SA), polyethylene glycols, polyorthoesters, polycarbonates, polyurethanes, polyacetals, polycyanoacrylates, polyphosphoesters, poly(oxyethylene), poly(oxypropylene), polydioxanes or polydioxanones, amino acid polymers such as polyarginines, and copolymers and blends of these polymers.

Preferred biocompatible polymers or copolymers are biodegradable polymers, more specifically polymers of lactic acid (PLA), polymers of glycolic acid, lactic acid-glycolic acid copolymers (PLGA).

The biodegradation of solid biodegradable polymers that preferentially contain lactic and/or glycolic acid releases lactic and/or glycolic acid, acids that potentially generate an acidic medium that can be conducive to the treatment of nail diseases and/or promote nail growth.

The compositions according to the invention may also contain other injectable excipients such as amino acids such as histidine, lysine or arginine, preferably arginine.

In other words, the first object of the present invention is a solid composition comprising one or more sustained-release biodegradable polymers, optionally other injectable excipients, and optionally one or more active substances for its use in the treatment of nail diseases and/or for promoting nail growth by subcutaneous administration at a distance greater than 1 cm from the edge of the nail on the side of its proximal end preferably in the finger with the exception of the last phalanx and preferably in the penultimate phalanx.

The first object of the present invention is therefore in particular a solid composition comprising one or more sustained-release biodegradable polymers, optionally other injectable excipients, and optionally one or more active substances for its use in the treatment of nail diseases by subcutaneous administration at a distance greater than 1 cm from the edge of the nail on the side of its proximal end preferably in the finger with the exception of the last phalanx and preferably in the penultimate phalanx.

The object of the present invention is therefore to facilitate the treatment of nail diseases and in particular the treatment of local mycoses. In particular, the object of the invention is to propose a loco-regional, internal, parenteral, i.e. injectable treatment of nail diseases of prolonged duration.

More specifically, the present invention may consist of new locoregional uses of new pharmaceutical forms with delayed or prolonged release that are highly concentrated in active substances but which correspond to low daily doses of antifungal agents compared to the doses delivered by the general systemic route or to the doses available with topical forms of locoregional use.

The present invention makes it possible, in particular, to treat fungal infections, in particular through the administration by internal subcutaneous injection at least 1 cm from the infected nail and outside the last phalanx which bears it, or by external insertion between the plate and the nail bed, of antifungal agents according to an unexpected and different dosage from the usual dosage, at very low doses and at very reduced frequencies, with superior efficacy.

Surprisingly, the inventors of the present application also discovered that the intervention which consists of injecting one or more implants subcutaneously, outside the site of the nail carrying the onychomycosis but in a place close to this site, allows effective treatment of nail disease and in particular onychomycosis without the need to administer not only the high doses of antifungals conventionally used in this type of treatment but also in the complete absence of active substances such as antifungals.

Depending on the type of nail disease, one or more of such implants which do not contain an active substance such as those envisaged in the compositions according to the invention may be administered at least 1 cm from the infected nail outside the phalanx which bears it and allow the treatment of the nail disease, in particular onychomycosis and alterations in the morphology of the nail or onychodystrophy.

It is then not necessary, but possible, to associate these medical devices in the form of implants containing no active substance, in particular anti-fungal agents which also constitute compositions according to the invention with one or more antifungal agents, whether they are administered near the diseased nail according to the invention or in a systemic manner.

However, it will be possible to incorporate a very low total dose compared to the doses necessary for these treatments of one or more of these molecules in order to avoid the contamination of the said implanted medical devices or the spread of mycosis. These very low doses of controlled release will also allow protection of the matrix when the nail infection progresses and threatens the nail matrix.

Although this attempt to explain the mechanism by which the devices constituting the compositions according to the invention and containing no or only very little active substance can cure onychomycosis may not constitute any limitation, it may be assumed that the implantation of such a device may lead to an inflammatory process and/or a micromechanical reaction to a foreign body with, in response, a mobilisation of the usual biological factors which will regenerate the implantation area and the surrounding tissues.

These processes (such as vasodilatation, the presence of macrophages) can also be triggered by an acidic environment linked to the processes of elimination of the biodegradable and slowly resorbable implanted device(s) made up, for example, of biodegradable polymers formed of lactic acid and/or glycolic acid, more commonly known as PLA or PLGA, which could result in mobilising the body's natural means of defence, elimination and/or healing, which could lead to the healing of the infected close nail.

Moreover, the implantable bioresorbable device creates, thanks to this micro-environment located close to the area to be treated, a barrier that also prevents superinfection, contamination or recurrence of colonisation by this micromechanical effect of a physical barrier that prevents the spread of the disease outside the infected area and creates protection.

The inventors of the present application have thus discovered that, in a surprising and advantageous way, nail diseases, and in particular onychomycosis, can be favourably treated by the implantation of a solid delay formulation under the skin, not implanted under the nail or in the immediately surrounding tissues, but at a certain distance from the diseased nail in sites that allow this type of injection, which is easier, more comfortable and non-pathogenic.

Moreover, the invention is the only solution that makes it possible to administer by injection or implant, i.e. by passing under the skin, in vascularised and innervated tissues, doses of active substances compatible with the needs of these prolonged treatments. It is also the only solution that allows access to the deep zone of the nail matrix by diffusing the treatment into the path of the vessels that feed and grow the nail.

In this way, the invention also offers the possibility of being used as a co-treatment with other therapies that have difficulty accessing this deep area of the matrix.

Surprisingly and unexpectedly, the invention offers the possibility, in a single administration, of releasing doses of active substances compatible with the need for treatment with an initial amount of treatment initiation as an example, which may, depending on the patient, correspond to very low doses of less than 10% of the total dose administered during the first week following administration, followed by periods of release at lower or higher levels depending on the grade of the disease and its rate of progression. Thus it is possible, depending on the invention, to cover different release regimes according to the needs of patients.

Treatment for the purposes of this application includes the prevention, protection, control, treatment or mitigation of a disease. It also includes treatment at the birth or deep root of the nail constituted by the womb, reducing the risk of recurrence, slowing the spread, stopping the spread of the disease and relieving pain. Indeed, the present invention offers the unique possibility of protecting the nail matrix as a fully-fledged characteristic of the treatment associated or not with the eradication of the infection which is or is not already present there is of great interest for all populations, populations already under oral or topical treatment, populations at high risk of infection or recurrence due to their pathological conditions or populations at high risk due to their daily sports activities or suffering from non-mycological alterations in the morphology of the nail.

The treatment of diseases of the nail within the meaning of the present invention also includes the acceleration of the growth of the healthy nail, in particular in the case of onychomycosis, or the restoration of this growth in the case where the disease of the nail, in particular onychomycosis, has totally or almost totally interrupted this growth. The restoration of an aesthetically and morphologically healthy nail is also one of the two compulsory clinical criteria established by the person skilled in the art to ascertain healing and the absence of mycological contamination and included in the international regulatory recommendations.

Indeed, one of the major problems with the effectiveness of treatments for nail diseases is related to the slow growth of nails, which is even slower in the case of diseased nails. The purpose of the invention is therefore to be able to use an excipient or an active substance capable of restoring and/or accelerating the growth of the healthy nail alone or in combination with other excipients or other active substances.

The present invention also has as its object the acceleration of the growth of the healthy nail in particular in the absence of onychomycosis.

The invention according to the present application differs from the prior art by two essential characteristics:

On the one hand:
either they do not contain any particular active substance at all, and in the case of the treatment of onychomycosis, they do not contain an antifungal agent,
either they contain only a very low total dose of active substance, in particular of antifungal agent, a dose only intended to avoid the contamination of the said implanted compositions or to protect the nail matrix or to avoid the propagation of mycosis, for example less than 50%, preferably less than 20%, more preferably less than 5%, more preferably less than 2%, even more preferably less than 1%.
or they contain a dose that is significantly lower than the dose usually used for treatment, particularly the treatment of onychomycosis with antifungals, e.g. less than 50 mg per area to be treated.

On the other hand, the present invention makes it possible to treat affections by fungi, in particular by administering at least 1 cm from the infected nail.

Antifungal agents, when present, are in unexpected and different dosages from the usual dosage, especially at very low doses and with superior efficacy.

In the case of terbinafine, for example, the prescribed dosage for oral forms for the treatment of nail diseases is generally 250 mg per day for 6 to 12 weeks, depending on the infected area, representing a total dose of 21 g of active substance over a 12-week treatment period, with results of only 38% total healing in clinical studies.

With the same active substances, the compositions according to the invention allow prolonged treatments over 48 weeks and more, with total doses of terbinafine inferior to 2 g, that is to say at least 10 times less, preferably less than 200 mg i.e. 100 times less, more advantageously less than 60 mg i.e. 350 times less, more advantageously still less than 20 mg per year i.e. at least 1000 less, and even more advantageously, less than 5 mg per year i.e. at least 4000 times less than those of the current oral forms.

Some active substances such as itraconazole can be prescribed for 48 weeks at a dose of 200 mg per day for results not exceeding 15% of patients who are fully treated with 67 g of active substance administered systemically.

In all the cases previously mentioned, the doses administered by the compositions according to the invention may be added to or combined with these treatments in order to improve the percentages of cure.

The present application also relates to the treatment of diseases of the nails by compositions containing one or more active substances for the treatment of diseases of the extremities, and in that these compositions according to the invention containing different active substances may be administered alternately in time and at the most suitable frequency. For example, it will be possible to administer during a first treatment period a composition containing an active substance such as terbinafine, followed by a second treatment period where the patient will receive a composition containing a second active substance, for example ciclopirox, which can be followed by a third treatment period where the patient can be re-administered with a composition again containing the first active substance, or a composition containing a third active substance, e.g. ciclopiroxolamine or itraconazole, until complete recovery. It is also possible to administer compositions containing several active substances at the same time, and to alternate them by treatment periods with compositions that may contain one or more different active substances.

Similarly, it is also possible to administer one or more implants at the same time, each containing one (or more) different active substances, and then alternate with one or more implants at the same time, each containing one (or more) active substances, which may or may not be different from the first active substances implanted.

On the other hand, the compositions according to the invention are administered, not systemically under the nail or in the immediately surrounding tissues, but at a distance of more than 1 cm from the edge of the nail on the side of its proximal end and preferably outside the last phalanx or a joint area, preferably in the penultimate phalanx.

The delivered doses of excipients and active substances are proportional to the total size of the solid formulations injected.

These new areas of administration make it possible to deliver higher daily doses than those compatible with the prior art from formulations of larger volumes, easier, more comfortable and less painful than those made too close or in the traumatic and particularly sensitive areas of the nail.

For example, one or more solid formulations with a diameter greater than 0.5 mm and a length greater than 1 cm can be injected. Such sizes of solid formulations require a needle with a diameter greater than 0.90 mm for administration under the nail or into the immediately surrounding tissue. It is easy to understand that the needle would be incompatible with injections in these areas that are too small, and would be extremely painful for the patient.

It is therefore easy to see that the shapes and volumes envisaged in the compositions according to the invention would in no way be compatible with the areas of administration of the treatment approaches proposed in the prior art.

The present invention thus has as its object compositions for their use in the treatment of diseases and/or alterations in the morphology of the nail and/or for promoting nail growth, and in that these compositions comprise one or more active substances. In particular, the present invention relates to a composition comprising one or more active substances chosen from:
Infectious antis selected from:
   antifungal agents,
   antibiotics
   and pest control products
Anti-inflammatory drugs selected from
   steroidal anti-inflammatory drugs
   non-steroidal anti-inflammatory drugs, and
   Janus Kinase inhibitors (JAK),
The cyclosporins,
vitamin D or vitamin D derivatives,
Immunosuppressants,
Amino acids, preferably Arginine,
Active substances that accelerate nail growth, preferably selected from:
   prostaglandins
   Chitosan, hydroxypropyl chitosan
   Valproic acid
   Echisetum arvense (horsetail), Hyaluronic Acid, Biotin, Growth Hormone (GH), Minoxidil, Finasteride.
Active substances promoting blood circulation chosen from among
   vasodilators and
   anti-platelet aggregants,
Local anaesthetics,
   for its use in the treatment of nail diseases by subcutaneous administration at a distance of more than 1 cm from the edge of the nail on the side of its proximal end preferably in the finger with the exception of the last phalanx.

The present invention therefore has as its object a composition as described above and preferably comprising one or more active substances chosen from among:
Antifungal agents, selected from Nystatin, Amphotericin B, Abafungin, Benzalkonium chloride, Caspofungin, Cetrimide, Clioquinol, Copper sulphate, Haloprogin, Echinocandins, Flucytosine, Mycobactovir, Novexatin, Natamycin, Cetylpyridium chloride, Benzylamine butenafine, Benzoxaboroles (cyclic derivatives of boronic acids), Albaconazole, Arasertaconazole, Bifonazole, Butoconazole, Clotrimazole, Eberconazole, Econazole, Eficonazole, Fenticonazole, Fluconazole, Fosravuconazole, Imidazole, Isoconazole, Irtemazole, Isavuconazole, Ketoconazole, Liarozole, Lianozole, Luliconazole, Miconazole, Oxadiazole, Oxiconazole, Posaconazole, Pramiconazole, Ravuconazole, Sertaconazole, Sulconazole, Terconazole, Thiazole, Thiabendazole, oteseconazole, Thiadiazole, Thiamazole, Tioconazole, Voriconazole, Arginine, Amiodarone, Naftifine, Tavaborole, Butenafine, Flucytosine, Viabecline, Griseofulvin, Caspofungin, Micafungin, Nitric Oxide, Sodium Oxychlorosene, Povidoneiodine, Piroctone olamine, Climbazole, Thiocarbonate tolnafate, Sulbentine, Zync Pyrithione, and more particularly among Terbinafine, Ciclopirox, Ciclopiroxolamine, Itraconazole, Miconazole, and Amorolfine.

Preferably, antifungals are selected from terbinafine possibly in the form of hydrochloride, ciclopirox, ciclopirox olamine, amorolfine and Miconazole.

In particular for the treatment of pseudomonas infections, the compositions according to the invention may contain an antibiotic, preferably Fluoroquinoles and tetracyclines,

The compositions of the invention may also include antiparasitic agents which may be selected from Emamectin, Selamectin, Imidacloprid, moxidectin, Milbemycin oxime, Lufenuron, Fumagillin, Antiamoebics: Rifampicin, Amphotericin B; Antihelmintics: Fenbendazole, Triclabendazole, Flubendazole, Abamectin, Suramin, Levamisole, Niclosamide, Nitazoxanide, Oxyclozanide, Aminoacetonitrile derivatives, Spiroindoles, Pelletierine sulphate and Artemisinin
for the treatment of mycoses and in particular onychomycosis, it may be advantageous to administer, using the compositions of the invention, an anti-inflammatory at the same time as one or more of the above-mentioned antifungal products.

Steroidal anti-inflammatory drugs that can be given at the same time as antifungals can be selected from the following: Hydrocortisone, Methylprednisolone, Prednisolone, Prednisone, Amcinonide, Budesonide, Desonide, Fluocinolone acetonide, Fluocinonide, Halcinonide, Beclometasone, Betamethasone, Fluocortolone, Halometasone, Mometasone, Alclometasone dipropionate, Betamethasone dipropionate and valerate, Clobetasol propionate and butyrate, Fluprednidene acetate, Mometasone furoate, Ciclesonide, Cortisone acetate, Hydrocortisone as aceponate, acetate, buteprate, butyrate and valerate, Prednicarbate, and Tixocortol pivalate, preferably Dexamethasone or Triamcinolone.

Non-steroidal anti-inflammatory drugs that can be given at the same time as antifungals can be selected from the following: Acetylsalicylic Acid and Lysine Acetylsalicylate, Methyl Salicylate, Diflunisal, Arylalkanoic Acids, Profenes, Indolic Derivatives, Oxicams, CINODs, Sulfonanilides, Coxibs Group, Phenylbutazone, Niflumic Acid and Phenamic Acids.

Another type of anti-inflammatory medication that can be administered are Janus Kinase Inhibitors (JAK),
for the treatment of inflammatory diseases and in particular psoriasis, whether or not accompanied by inflammatory phenomena, one of the above-mentioned anti-inflammatory drugs can also be used.

In addition to one or more anti-inflammatory drugs, the treatment of nail diseases and in particular the treatment of inflammatory diseases, such as psoriasis, can be carried out using compositions of the invention comprising one or more cyclosporins, preferably cyclosporin A, vitamin D or derivatives of vitamin D, preferably calcipotriol or paricalcitol,

The compositions of the invention may also include immunosuppressants, preferably methotrexate, retinoids, preferably Tazarotene, or compounds such as Anthralin (or dithranol) and Urea.

The compositions according to the invention may also contain one or more amino acids, preferably Arginine.

The compositions of the invention may also include one or more active substances accelerating the growth of the nail, preferably selected from prostaglandins. The prostaglandins which may be used may be preferably selected from among the derivatives of the prostaglandin F2α or the pro-drugs of PGF2alpha, and more preferably latanoprost, travoprost or bimatoprost, even more preferably latanoprost or bimatoprost.

Chitosan, hydroxypropylchitosan, Valproic Acid, Echisetum arvense (horsetail), Hyaluronic Acid, Biotin, Growth Hormone (GH), Minoxidil and/or Finasteride can also be used to promote nail growth.

The compositions of the invention may also include one or more active substances promoting blood circulation such as vasodilators and anti-platelet aggregants. The vasodilators can be chosen from Amlodipine (besylate, mesylate or maleate), Nifedipine, Diltiazem, Verapamil, Ginkgo biloba, Iloprost, Endotheline, Nacitentan, Bosentan, Nicotinic Acid (Niacin), Arginine, Nitric Oxide, Nitroglycerine, Isosorbide Dinitrate, Prostacycline (PGI2) and other prostaglandins, Histamine, Bradykinin.
In particular for the treatment of complications of the diabetic foot, Buerger's disease, Raynaud's disease, arteritis of the lower limbs, an anti-platelet agent such as Clopidogrel can be used,

The compositions of the invention may also include local anaesthetics which may be selected from Lidocaine, Articaine, Bupivacaine, Lebobupivacaine, Mepivacaine, Procaine and Ropivacaine,

Preferably compositions containing valproic acid, which is a generally known substance used in the treatment of diseases caused by keratin deficiency such as androgenic alopecia, are used,

The present invention has in particular the object of a composition as described above for its use in the treatment of nail diseases, characterized in that the nail disease is onychomycosis and in that the composition comprises one or more of the above-mentioned active substances selected from:
antifungal agents preferably selected from terbinafine optionally in the form of hydrochloride, ciclopirox, ciclopiroxolamine, amorolfine and Miconazole, itraconazole
steroidal or non-steroidal anti-inflammatory drugs, preferably selected from dexamethasone and triamcinolone
cyclosporins,
amino acids, preferably Arginine,
active substances accelerating nail growth, in particular chitosan, hydroxypropylchitosan
vasodilators
local anaesthetics
Antibiotics
antiparasitic drugs, including emamectin, ivermectin and selamectin

The compositions according to the invention which are used to treat the above-mentioned inflammatory diseases and in particular psoriasis may advantageously contain one or more cyclosporins such as cyclosporin A or G, preferably cyclosporin A.

The present invention is therefore also particularly aimed at a composition as described above for its use in the treatment of nail diseases, characterized in that the nail disease is psoriasis accompanied or not by an inflammatory phenomenon and in that the composition comprises one or more active substances preferably chosen from:
steroidal anti-inflammatory drugs among: Hydrocortisone, Methylprednisolone, Prednisolone, Prednisone, Amcinonide, Budesonide, Desonide, Fluocinolone acetonide, Fluocinonide, Halcinonide, Beclometasone, Betamethasone, Fluocortolone, Halometasone, Mometasone, Alclometasone dipropionate, Betamethasone dipropionate and valerate, Clobetasol propionate and butyrate, Fluprednidene acetate, Mometasone furoate, Ciclesonide, Cortisone acetate, Hydrocortisone as aceponate, acetate, buteprate, butyrate and valerate, Prednicarbate, and Tixocortol pivalate, preferably Dexamethasone or Triamcinolone.
Non-steroidal anti-inflammatory drugs such as: Acetylsalicylic acid and Lysine Acetylsalicylate, Methyl Salicylate, Diflunisal, Arylalkanoic acid, Profenes, Indolic derivatives, Oxicams, CINODs, Sulfonanilides, Coxibs group, Phenylbutazone, Niflumic acid and Phenamic acids.
Janus Kinase inhibitors (JAK),
one or more cyclosporins, preferably cyclosporin A,
vitamin D or vitamin D derivatives, preferably calcipotriol or paricalcitol,
vasodilators,
immunosuppressants, preferably methotrexate,
retinoids, preferably Tazarotene,
compounds such as Anthralin (or dithranol) and Urea.
Active substances that accelerate nail growth, preferably latanoprost or bimatoprost, selected from prostaglandins;
chitosan, hydroxypropyl chitosan
valproic acid.
vasodilators
local anaesthetics
the Arginine

For the treatment of complications of the diabetic foot, Buerger's disease, Raynaud's disease, arteritis of the lower limbs, an anti-platelet agent such as Clopidogrel can be added to the composition according to the invention, as well as vasodilators, selected from Amlodipine (besylate, mesylate or maleate), Arginine, Nifedipine, Diltiazem, Verapamil, Ginkgo biloba, Iloprost, Endotheline, Nacitentan, Bosentan, Nicotinic Acid (Niacin), Nitric Oxide, Arginine, Nitroglycerine, Isosorbide Dinitrate, Prostacycline (PGI2) and other prostaglandins, or for example Histamine and Bradykinin.

When only the growth of the nail is sought, the compositions according to the invention may contain one or more active substances chosen from among the cyclosporins, prostaglandins, preferably the cyclosporins and prostaglandins mentioned above, chitosan, hydroxypropylchitosan and/or valproic acid.

The present invention also has as its object in particular a composition as described above for its use to accelerate the growth of the nail characterized in that it comprises one or more active substances chosen from:
cyclosporins, preferably cyclosporin A,
prostaglandins preferably selected from latanoprost or bimatoprost,
Echisetum arvense (horsetail), Chitosan, hydroxypropyl chitosan, Hyaluronic acid, Biotin, Valproic acid, Growth hormone (GH), Minoxidil and/or Finasteride.
amino acids, preferably Arginine,
vasodilators, such as gingko biloba, nitric oxide and arginine
valproic acid.

The present invention is also particularly aimed at a non-therapeutic method of accelerating the growth of the nail, characterized in that it comprises the administration of a composition as defined above and comprising one or more solid biodegradable sustained-release polymers, one or more active substances chosen from among:
cyclosporins
prostaglandins
Echisetum arvense (horsetail), Chitosan, Hydroxypropyl chitosan, Hyaluronic acid, Arginine, Biotin, Valproic acid, Growth hormone (GH), Minoxidil and/or Finasteride.
Vasodilators
valproic acid.
and possibly one or more active substances selected from:
Anti-infectives selected from:
   antifungal agents,
   Antibiotics
   and pest control products
Anti-inflammatory drugs selected from
   steroidal anti-inflammatory drugs
   non-steroidal anti-inflammatory drugs, and
   Janus Kinase inhibitors (JAK),
vitamin D or vitamin D derivatives,
Immunosuppressants,
Amino acids, preferably Arginine,
Active substances promoting blood circulation chosen from among
   vasodilators and
   anti-platelet aggregants,
Local anaesthetics,
   and in that it is in the form of a device intended to be administered subcutaneously at a distance greater than 1 cm from the edge of the nail, preferably in the finger, except for the last phalanx.

The present invention is also particularly aimed at using a composition as defined above to accelerate the growth of the nail.

In summary, for the treatment of mycosis and in particular onychomycosis, it may be advantageous to administer, using the compositions of the invention, an anti-inflammatory, preferably a steroidal anti-inflammatory, at the same time as one or more of the above-mentioned antifungal products.

A composition according to the invention may preferably include:

One or more antifungal products selected from the following products:

Ciclopiroxolamine, Amorolofine, Imidazoles, Tolnaftate, Bifonazole, Butenafine, Tioconazole, Terbinafine, Itraconazole, Fluconazole, Griseofulvin, Ketoconazole, Ciclopiroxamine, Ciclopirox, Miconazole and more particularly among Terbinafine, Ciclopirox, ciclopiroxolamine, itraconazole and amorolfine.

One or more anti-inflammatory drugs that can be given at the same time as the antifungals are preferably selected from:
Steroidal anti-inflammatory drugs among: Hydrocortisone, Methylprednisolone, Prednisolone, Prednisone, Amcinonide, Budesonide, Desonide, Fluocinolone acetonide, Fluocinonide, Halcinonide, Beclometasone, Betamethasone, Fluocortolone, Halometasone, Mometasone, Alclometasone dipropionate, Betamethasone dipropionate and valerate, Clobetasol propionate and butyrate, Fluprednidene acetate, Mometasone furoate, Ciclesonide, Cortisone acetate, Hydrocortisone as aceponate, acetate, buteprate, butyrate and valerate, Prednicarbate, and Tixocortol pivalate, preferably Dexamethasone or Triamcinolone.
Non-steroidal anti-inflammatory drugs among: Acetylsalicylic Acid and Lysine Acetylsalicylate, Methyl Salicylate, Diflunisal, Arylalkanoics, Profenes, Indolic derivatives, Oxicams, CINODs, Sulfonanilides, Coxibs group, Phenylbutazone, Niflumic Acid and Phenamic Acids.
Janus Kinase (JAK) inhibitors,

One or more vasodilators, which can be chosen from Amlodipine (besylate, mesylate or maleate), Nifedipine, Arginine, Diltiazem, Verapamil, Ginkgo biloba, Iloprost, Endotheline, Nacitentan, Bosentan, Nicotinic Acid (Niacin), Nitric Oxide, Nitroglycerin, Isosorbide Dinitrate, Prostacyclin (PGI2) and other prostaglandins, Histamine, Bradykinin,

A local anesthetic such as lidocaine.

The compositions according to the invention may take any shape or size which is compatible with the specific new areas of administration and with the triggering of a local reaction of the organism which is capable of treating the nail disease in the absence of an active substance such as an antifungal agent or the ability to deliver the sufficient quantity of active substance to be therapeutically effective in the treatment of the nail disease and/or the acceleration or restoration of the growth or regrowth of the nail.

The compositions can be in the form of an implant, possibly with a sharpened end, or in the form of a cylindrical tube, bar or wire. They can also be in the form of particles, discs or sheets.

The compositions of the invention can, for example, be administered by means of a needle or a device which completely avoids the use of a needle and with which the compositions are injected directly under the patient's skin. Devices of this type, the compositions that can be administered in this way and their method of preparation are, for example, described in international application WO 96/08289.

The compositions according to the invention may for example be comparable to a mono- or multi-filament suture suture of PLA and PLGA, preferably with a length of at least 10 mm and up to 25 mm. These flexible threads can be applied with a needle and have the advantage that they can be adapted to the shape of the finger.

According to the different forms that the compositions of the invention may take (implants, wires) one can implant, for example, two or more implants or wires on each side of the finger, possibly symmetrically and preferentially on the external dorsal parts of the penultimate phalanx.

In the form of threads, compositions of the invention may have a greater length, greater than 25 mm, and for example 10 cm, so that they can be implanted by cutting them to the desired lengths, from the same needle, for example on either side of the finger to be treated or one next to the other in the same finger.

Another advantageous presentation of the compositions according to the invention in the form of threads fixed directly to a needle having a curvature will allow their introduction under the skin and its exit after insertion of a length which can be variable according to the needs of treatment and according to the morphology of the fingers.

According to this advantageous administration procedure, during administration, it is sufficient to carry out the implantation until the end of the thread opposite the needle disappears under the skin and then to cut this thread on the surface of the skin. Implantations of these thread sections from the same thread and needle can be repeated. Another advantage of this device is that it allows implantation with a needle diameter comparable to that of the thread and not larger as in the case where the thread is previously contained inside the needle. This reduces pain, improves the comfort of the treatment as well as its maintenance in situ in tissues in direct contact with the thread.

The compositions can advantageously have a pointed tip in the shape of a toothpick and have sufficient hardness to allow them to penetrate through the skin into the subcutaneous layers.

The present invention also particularly relates to a composition as described above for its use in the treatment of nail diseases and/or to accelerate the growth of the nail, characterized in that it is in the form of an implant, a cylindrical tube or a thread, optionally with a sharpened end, and in that the composition is administered in the finger with the exception of the last phalanx. According to the present invention, “accelerating the growth of the nail” or “accelerating the growth of the nail” means restoring, allowing and/or increasing the speed of the growth of the nail.

According to a preferred mode of execution of the invention, the compositions according to the invention take the form of implants comprising one or more active substances and one or more polymers, which are biocompatible, biodegradable or bioresorbable.

Biocompatible means a polymer that does not cause irritation to the surrounding tissue.

By biodegradable we mean polymers that degrade over time under the action of enzymes or hydrolyses.

Bioabsorbable or bioresorbable means that the polymeric component(s) of the compositions are metabolised and are absorbed by the surrounding tissues.

In a preferred mode of realization of the invention, copolymers of lactic and glycolic acid (PLGA) are used. The percentage of each monomer can vary from 0 to 100%. For example, it can be about 50/50 but can also be about 100-0%, 85-15%, 75-25%, 65-35%, and 55-45% or less.

Polymers and copolymers can be capped at their ends with an acid or ester function.

Preferred solid sustained release polymers are polymers of L-lactic acid, D-lactic acid, DL-lactic acid, L-lactide, D-lactide, DL-lactide, glycolide, glycolic acid, caprolactone, poly(dioxanone) and all corresponding copolymers.

The preferred PLA polymer is a poly (D,L-lactide) with a viscosity between 0.16 and 2 dL/g, preferably 0.20 to 1.8 dL/g and preferably 0.5 to 1.2 dL/g.

The preferred PLGA copolymer is an 85:15 with a viscosity between 0.15 and 7 dL/g, preferably in the range of 0.25 to 3 dL/g, and preferably in the range of 0.50 to 1.8 dL/g.

The active substance content can vary between 0 and 99%, in particular be less than 85%, preferably less than 75%, preferably between 20 and 70%. It may in particular be 20, 30, 35, 40, 50, 51, 52, 53, 54, 55, 56, 57, 58, 59, 60, 61, 62, 63, 64, 65, 66, 67, 68, 69 or 70%.

As indicated above, the compositions according to the invention may not contain any active substance at all (“placebo” composition). They may also contain a very low dose of active substance, for example an antifungal agent, to avoid contamination of the implanted compositions, to protect the area or to prevent the spread of fungus. In the latter case, the concentration may be of the order of 0.01%.

The quantity of active substance, preferably Terbinafine hydrochloride, ciclopiroxolamine or Ciclopirox, itraconazole per implant which is the preferred form of the compositions of the invention may be between 0 and 6.5 mg, more particularly between 0.01 and 2.5 mg of product. This amount of active substance may be, for example, 0.01, 0.02, 0.04, 0.16, 0.20, 0.30, 0.5, 0.78, 1.00, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, 5.00, 5.10, 5.20, 5.30, 5.40, 5.50, 5.60, 5.70, 5.80, 5.90, 6.00, 6.10, 6.20, 6.30, 6.40 or 6.50 mg.

The present invention has in particular the object of a composition as described above, characterized in that the solid polymer with prolonged release is a polyester, preferably a lactic acid-glycolic acid copolymer (PLGA) or a polymer of lactic acid (PLA), and in that the content in active substance is less than or equal to 85% and preferably between 20 and 70% by weight.

The present invention has in particular the object of a composition as described above characterized in that it comprises between 0.04 and 6.50 mg of terbinafine hydrochloride or terbinafine and PLGA.

The present invention has in particular the object of a composition as described above characterized in that it comprises between 0.04 and 6.50 mg of ciclopirox or ciclopiroxolamine and PLGA.

The present invention relates in particular to a composition as described above characterized in that it comprises between 0.04 and 6.50 mg of itraconazole and PLGA.

The compositions according to the invention may have a total volume per implant or injection of not more than about 30 mm³, their volume may be between about 5 and about 30 mm³, preferably between about 10 and about 30 mm³. They may, however, have a total volume of more than 30 mm³.

The diameter of the implant can be between 0.1 and 1 mm, preferably between 0.1 and 0.9, more preferably between 0.3 and 0.8 mm and in particular the diameter can be 0.1, 0.25, 0.3, 0.4, 0.45, 0.50, 0.55, 0.56, 0.57, 0.58, 0.59, 0.60, 0.65, 0.70, 0.75, 0.85 or 0.90 mm.

The length of the implant by injection can be a maximum of 25 mm, preferably between 4 and 15 mm, for example 4, 6, 8, 9 or 10 mm. The total length can be more than 25 mm and the implant can be cut to the measurements required for each implantation.

The object of the present invention is therefore a composition for its use in the treatment of nail mycoses, also called onychomycosis, characterized in that it comprises one or more solid biodegradable sustained-release polymers, and optionally one or more antifungal agents, and in that it is in the form of an implant having a maximum injected length of 25 mm, preferably between 4 and 15 mm, and a diameter of between 0 and 0.1 and 1 mm preferably between 0.3 and 0.8 mm, implant intended to be administered subcutaneously at a distance greater than 1 cm from the edge of the nail, preferably in the finger, except for the last phalanx.

In other words, the object of the present invention is therefore a composition in the form of an implant having a maximum injected length of 25 mm preferably between 4 and 15 mm and a diameter of between 0.1 and 1 mm preferably between 0.3 and 0.8 mm comprising one or more solid biodegradable sustained-release polymers and one or more antifungal agents for use in the treatment of onychomycosis by subcutaneous administration at a distance greater than 1 cm from the edge of the nail towards its proximal end preferably in the finger with the exception of the last phalanx.

The invention therefore also has as its object, a composition described above for its use in the treatment of nail diseases and/or to accelerate the growth of the nail, characterized in that it is in the form of an implant, a cylindrical tube or a thread, possibly with a sharpened end, and in that the composition is administered in the finger with the exception of the last phalanx, in the first or second phalanx of the fingers, preferably the second phalanx and in the first phalanx of the thumb or big toe and in that one or more compositions, preferably from one to eight compositions, more preferably from one to six compositions are administered simultaneously in the same finger.

The invention has especially for object the composition described above characterized in that the treatment is continuous, preferably for a minimum duration of 48 weeks when the whole nail is affected, a minimum duration of 24 weeks when less than half of the nail is affected, or a minimum duration of 12 weeks when less than a quarter of the nail is affected, or a duration of 6 weeks, preferably less than 6 weeks when less than 10% of the nail is affected.

For the avoidance of doubt, it is specified that the compositions according to the invention may, when used in the claimed applications, contain one or more active substances selected from any of the above-mentioned active substances.

It is possible to mix different active substances in the same implant or thread. It is also possible to make implants or threads with each active substance and to combine the administration of two or more of these implants and threads according to the specific needs of each patient, for example according to the types of fungi involved in their mycosis, or to treat the inflammation related to the mycosis at the same time as the fungus causing the mycosis.

The administration of the compositions according to the invention shall be carried out subcutaneously. As indicated above, a suitable device may be used to carry out this administration and an example of such a device is described in international application WO 96/08289.

The invention therefore has as its object a composition as described above for its administration by subcutaneous route in the first or second phalanx of the fingers, preferably the penultimate phalanx, i.e. the second phalanx, and in the first phalanx of the thumb or big toe.

The compositions according to the present invention are therefore administered or injected away from the fingertip and at a distance of more than 1 cm from the cuticle, and in particular they are administered subcutaneously outside the distal phalanxes, in the intermediate or proximal phalanxes or, in the case of the feet, in the proximal phalanxes or first or second phalanxes of the fingers, preferably the second phalanx and in the first phalanx of the thumb or big toe.

Preferably these compositions are administered on the dorsal lateral parts of the fingers or on each side of the dorsal and not palmar faces of the penultimate phalanx, in the middle of this phalanx at an equal distance from the two joints which delimit it.

The novel and inventive character of the present invention lies in particular in the fact that the administration or injection of the compositions according to the invention is not carried out under the nail or in the immediate vicinity of the nail as is practiced with known injections for these types of treatments and as has been proposed in several prior art applications, in particular in the following documents: U.S. Pat. No. 8,747,820, international applications WO2011/087867 or WO2006/063350 but at a distance greater than 1 cm from the proximal end of the nail outside the phalanx which bears it.

As indicated above, administration or injection into living tissue, outside the diseased area of the nail or its immediate environment avoids disturbing an area traumatised by the fungus, a disturbance that delays healing. Furthermore, this unaffected area is larger and it is easier and much less painful to inject there than in the nail area and this space allows for the administration of several implants if necessary.

On the other hand, the small volume available for injection under the nail or in the immediate vicinity of the nail does not generally allow for the delivery of a sufficient dose of active substance for the treatment envisaged: this does not allow for targeting the base of the growth or renewal of the nail that constitutes the matrix.

The invention therefore has as its object a composition as described above and characterized in that one or more compositions, preferably from one to six compositions, more preferably from 1 to 4 compositions are administered simultaneously in the same finger.

This area upstream of the nail allows the injected treatments to be directed preferentially or in priority towards the matrix at the deep source of the nail, in an area that is also inaccessible to external topical forms. This makes it possible to envisage in-depth treatment of fungal infections, for example in the case of an infection of the whole nail or a significant part of the nail, without the disadvantages of oral forms and therefore over much longer periods of time until the needs can be covered after more than a year to avoid a recurrence or reinfection.

In order to carry out this injection or deposit and to avoid that this treatment is painful for the patient, a local anaesthesia, topical or injectable, can be previously performed on the subcutaneous implantation area.

The specific treatment of administration can be carried out immediately and quickly by a specialised dermatologist or chiropodist or authorised medical personnel depending on the territory. It avoids the patient any local treatment, for example with creams, a treatment often made difficult with age, long and complicated to apply to the patient. He ensures the follow-up of the treatment which no longer depends on the patient's compliance.

The deposit can be subcutaneous or intradermal.

Compared to LASER treatments sometimes involving nail piercing, the administration of the compositions according to the invention makes it possible to obtain a better result with far fewer constraints and at a much lower cost.

However, more recently, new methods of treatment for nail diseases and in particular onychomycosis have been developed. These treatments consist of causing the spores to break and the destruction of the fungus by the photobiological thermal effect of the nail plate by LASER. The treatment is generally to be repeated at least 3 times, leaving 4 to 6 weeks between each treatment.

During the LASER treatment, a LASER beam at a controlled wavelength is directed towards the nail bed, generating heat under the nail by thermal effect at a specific wavelength to destroy the fungal colony.

Results obtained with this technique have been reported for example in the following publication: Novel LASER Therapy in treatment of Onychomycosis, Jasmina Kozarev et al. Journal of the Laser and Health Academy, Vol. 2010, No. 1; p. 1

Several commercial LASER beam generators can be used, the devices PinPointe™ FootLASER™ or ND:YAG or in particular the S30 PODYLAS™ device. Commonly used wavelengths are 870, 930 or especially 1064 nm.

However, these LASER treatments do not always reach deep infected areas and therefore may be limited in their effectiveness and expensive, perhaps a limitation to their use. Furthermore, the effectiveness of the treatment is directly dependent on the practitioner's adherence to the strict protocol of LASER application.

The compositions according to the invention can therefore be usefully combined with the LASER treatment to reach deep infected areas inaccessible to the LASER and improve its effectiveness.

It is clear that although repeated injections can be envisaged for these treatments, the most suitable approach is that of a single injection or a minimum of injections which will then release the active substance(s), particularly antifungal ingredients, locally over a sufficient period of time, compatible with the full treatment, preferably several months to a year.

Advantageously, it is therefore possible, thanks to the invention, to administer once a year, half-yearly, quarterly, monthly or weekly, depending on the severity of the infection, a dose of 0 to 50 mg of active substance per affected area, or of different active substances.

It is possible to multiply the treatments and administrations for the same treatment and thus best cover the needs of patients according to the type of fungus and other concomitant nail diseases without administering high doses or the usual therapeutic doses used orally or topically.

It is also possible, depending on the needs of the patients and their pathology, to adapt an adapted release profile both in terms of doses and quantities released over time in a single or multiple doses.

Preferably, the administration of the active substance(s) using the compositions of the invention is carried out for at least 48 weeks. Preferably this administration shall be carried out for more than 48 weeks.

Preferably, this administration replaces the usual high-dose oral treatment at the beginning of severe fungal infections and can be continued throughout the treatment until recovery even after one year or when other treatments, e.g. topical treatments, are interrupted, to avoid reinfection of the matrix and the risk of recurrence and to protect it during the progression of distal onychomycosis.

The purpose of the invention is therefore a composition as described above for use in the treatment of nail diseases, characterized in that one or more compositions are administered simultaneously in the same finger and in that the treatment is continuous, preferably for a minimum period of 48 weeks. Preferably the administration takes place monthly, quarterly or every six months, and the treatment is delivered over at least twelve months or even eighteen months.

Another object of the invention is to treat certain diseases of the nail in which the entire nail is not affected, but only part of it is infected or diseased. In these specific cases, the duration of treatment will be less than 48 weeks; for example, the minimum duration of treatment may be 24 weeks if less than half of the nail is affected by the disease, or the minimum duration of treatment may be 12 weeks if less than a quarter of the nail is affected by the disease, or less than 12 weeks if less than one tenth of the nail is affected by the disease or susceptible to infection.

The purpose of the invention is therefore a composition as described above for use in the treatment of nail diseases characterized in that one or more compositions are administered simultaneously in the same finger and in that the treatment comprises one or more intervals during which the treatment is delivered from these punctual administrations.

Subcutaneous injection administration of implants or wires can be performed with solid compositions contented inside hollow needles of larger diameter than implants or threads. These administrations can thus be performed with solid compositions attached to the other end of a solid needle of the same diameter as the implants or threads from a single needle.

Compared to oral treatments, one of the other interests of this approach to treatments according to the invention, and in particular antifungal treatments, is to limit the quantity of active products in the whole body in areas where they are undesirable and also to considerably reduce the total quantity of product used for the same treatment.

This also makes it possible to control the risks of environmental contamination with the emerging molecules from drug rejects and waste, which constitute a serious, growing and difficult to avoid threat, much better controlled upstream in the approach according to the present invention, with quantities up to 1000 or even 5000 times less of these molecules used for the same treatment, 100% of the useful drug dose and no product disposed of without being used by the patient, as is often the case with the remains of tablets or liquids in bottles of the usual treatments.

One of the other interests of the treatment approach according to the invention obtained by delayed or controlled compositions that release their active substances in a prolonged manner over time is to allow the active substance(s) to be permanently present at their sites of action. It is also known that some of these molecules are likely to accumulate in the nail, which contributes to their effectiveness in the uses according to the invention and to the possibility of reducing the doses.

The invention therefore also relates to the uses of these compositions associated with other treatments such as oral, topical or LASER treatments to ensure in particular that infections are treated at the deep level of the matrix to ensure that there is always the necessary treatment in situ, or to prolong the treatment after these treatments to avoid recurrences or to protect the area during an infection with rapid progression or a population at high risk of complications.

Another object of the invention is to offer an external loco-regional treatment of prolonged duration of nail diseases based on these same solid compositions, by insertion between the plate and the nail bed when they are separated or easily separable.

The inventors thus discovered that these diseases can also be treated externally by inserting between the plate and the nail bed one or more solid compositions depending on the invention, which have major advantages. On the one hand, it offers the advantage of easy and precise localization due to its solid monolithic form of controlled shape and size that can be easily manipulated for insertion through the networks and meanders of filamentous hyphae of fungi. Furthermore, once in contact with infected tissues under the influence of ambient humidity or frequently brought to this level, the composition will quickly adapt to the available volumes by becoming flexible and softening, thus avoiding the creation of a solid physical barrier that could hinder the regrowth of a healthy nail. In fact, the compositions according to the invention become flexible to the point of becoming liquid or semi-solid when they are in contact with aqueous media for a prolonged period of time, a preliminary step in the biodegradation of the polymers that compose them.

The present application is therefore also for a solid composition for use in the treatment of nail diseases and/or for promoting nail growth comprising one or more solid polymers, biodegradable or not, with prolonged release, and optionally one or more active substances, and in that it is in the form of a device intended to be administered by insertion into the space between the bed and the nail plate and in that it is optionally used simultaneously or spread out over time with subcutaneous administration at a distance greater than 1 cm from the edge of the nail on the side of its proximal end preferably in the finger with the exception of the last phalanx of one or more of the same compositions described above and comprising one or more solid biodegradable sustained-release polymers, and optionally one or more active substances.

Depending on the case, this inserted external approach may be used either separately or in combination with the implanted internal use of the solid compositions of the invention for the same result.

These two therapeutic approaches, internal and external delay, may be combined and release one or more active substances in the same formulation or in different formulations administered separately according to the needs of each patient.

The inventors have discovered that this type of delay formulation when inserted in a less irrigated area with less exchange because it is not vascularised like the subcutaneous tissue, allows a much longer delay effect to be obtained even with the active substances being maintained in situ. It is therefore conceivable that the same formulation, which for example needs to be renewed every 3 months when implanted under the skin, can produce a therapeutic effect over a year when inserted above the nail bed. This type of external treatment can therefore be carried out only once, and can be continued until complete healing.

At the nail bed, in an area that can be separated from the corneal plaque by the fungal infection or in the plaque itself thickened by the fungus, and which is difficult to access by external topical treatments, it will be possible to combine a treatment by insertion of the same active substances. Advantageously, the solid composition according to the invention has the particularity by its composition, size and rigidity to be able to be inserted with or without a delivery device in the subungual space between the nail and the nail bed.

An infected nail often experiences this lifting and thickening of the corneal plate from the nail bed, called onycholysis and onychodystrophy. This onycholysis and onychodystrophy is due to an overly rapid renewal of the nail bed cells caused by an inflammatory response to the fungal infection. This onycholysis can be advantageous for the administration of formulations containing anti-infective actives by creating a space between the nail and the nail bed to allow a more efficient release of the active directly into the infected area, while remaining non-invasive and therefore painless.

Moreover, this onycholysis can also be the site of attack of the fungal infection preferred in the case of onychodystrophy not initially directly linked to a mycological infection. It is also advisable, when possible, to protect the affected nail by preventive treatment.

In particular, the inventors discovered that the composition can, without the need to create a prior channel using a sharp perforating tip, be pushed directly into the free space without creating trauma to the area to be treated and without the use of tools that could create tissue injury or an overly large opening with unnecessary space that could lead to greater exposure to external sources of contamination and aqueous environments.

In particular, the inventors discovered that if an instrument is used to insert the composition it is ideally equipped with a blunt, non-sharp end. It can be hollow and contain within it the composition to be back deposited using a piston or mandrel at a specific location. It can also be solid and thus allow the creation of a passage channel adjusted to the dimensions of the composition in the middle of the residues and hyphae of fungi infiltrating the space.

The inventors have discovered that one or more compositions to cover the entire surface to be treated according to the invention thus inserted and deposited remain maintained in a prolonged manner in space and allow by their system of prolonged release and softening a total direct exposure of the hyphae and filaments of the fungi to the therapeutic antifungal agents accompanying without preventing the growth of the nail, thus creating a mobile antifungal reservoir but without contact with the outside.

These external delay treatments according to the invention do not require daily applications but weekly or monthly or quarterly or semi-annual or annual applications as the new injectable treatments.

They can be administered to patients at the same time and with the same spaced frequency as injectable internal treatments. They may also be administered less frequently than internal treatments.

The present invention therefore also concerns one or more solid compositions according to the invention for their use in the treatment of nail diseases and/or for promoting nail growth comprising one or more biodegradable or non-biodegradable sustained-release polymers, and one or more active substances, characterized in that they are in the form of a device intended to be administered by insertion into the empty or prepared spaces between the bed and the nail plate and in that they are optionally used simultaneously or spread out over time with administration by subcutaneous route at a distance greater than 1 cm from the proximal end of the nail preferably in the finger with the exception of the last phalanx of one or more solid compositions as described above and comprising one or more biodegradable polymers.

In other words, the present invention therefore also concerns a solid composition comprising one or more monomers or polymers, biodegradable or not, with prolonged release and one or more antifungals for its use in the treatment of nail diseases by administration by direct insertion into the empty spaces or previously pushed into a canal of the dimensions of the implant previously created in the networks of hyphae and debris present between the bed and the nail plate, optionally simultaneously or spread out in time with subcutaneous administration in the finger at a distance greater than 1 cm from the proximal end of the nail, preferably with the exception of the last phalanx of one or more solid compositions as described above and comprising one or more biodegradable polymers.

Another object of the invention is to offer an external loco-regional treatment of prolonged duration with other liquid or non-solid, delayed or controlled compositions of nail diseases by insertion between the plate and the nail bed when they are separated or easily separable. The term non-solid composition includes liquid compositions.

The inventors also discovered that these diseases can also be treated externally by inserting between the plate and the nail bed a delayed or controlled formulation, liquid or non-solid, which will fit the available volumes and not create a solid physical barrier that could hinder the regrowth of a healthy nail.

Depending on the case, this approach may be used either separately or in combination with the use of the solid compositions of the invention for the same result.

These two forms of internal and external delay may possibly combine and release one or more active substances in the same formulation or in different formulations administered separately according to the needs of each patient.

At the level of the nail bed, in an area that can be separated from the hardened plaque by the fungal infection or in the plaque itself thickened by the fungus, and which is difficult to access by external topical treatments, it will be possible to combine treatment by insertion or infiltration of a suspension of the same active substances in a liquid or non-solid form, which can be polymerisable, solidifiable or not by different means (heat, light, solvents . . . ), made for example from monomers forming a gelled network in a solvent, aqueous or not, which will form a hydrogel, an organogel or any other gel that can contain and release active substances.

An example of a polymer(s) that can be used to contain the active substance(s) for this type of administration or external insertion with a prolonged or delayed effect may belong to the family of Polyhydroxyethyl methacrylates, hydroxyethyl methacrylates, carboxymethylcelluloses, hyaluronic acids, or any other polymer forming a hydrogel, biodegradable or not, in formulation alone, associated with each other or formulated with other excipients.

An example of non-delaying formulations for topical and through-the-skin/nail administration that can be used to contain the organogel-forming active substance is described in Mediquest Therapeutics' patents WO 2009/097471 and WO 2006/042059.

Another example of a polymer and associated formulations that can be used to contain the active substance(s) for external administration is described in Gecko Biomedical's patent WO2016202985, which may act as an adhesive that releases the active substance as it biodegrades. In this case, like the previous biopolymers, polymerisation can be carried out in-situ after administration.

Another example of formulations that may contain the active substance for external administration is described in Hallux's US patent 2010/0048724. Here the proposed area of administration is between the nail plate and the nail bed, but the compositions envisaged are not delayed and therefore involve frequent and repeated administrations which do not provide a satisfactory response for this area of treatment in which it is preferable not to intervene frequently.

Finally, the polymer containing the active substance in suspension may be in non-aqueous form and neither solid nor solidifiable, made for example from PLGAs according to patents WO2004/067602, WO2005/100439, WO2006/018524, WO2007/085729, WO2008/049631, WO2009/138589, WO2012/06619 and WO2012/066194. This formulation, which is not solid, contains no organic solvent and does not dry out, is likely to have a prolonged local effect without interfering with nail regrowth. These suspensions and treatments constitute another object of the invention.

Depending on the invention, these new delayed treatments do not require daily application but weekly or monthly or quarterly as the new injectable treatments.

They can be administered to patients at the same time and with the same spaced frequency as injectable internal treatments.

The internal treatment can be continued for as long as necessary, possibly in addition to and at the same time as the delayed external treatment by insertion at the nail plate or at the nail bed.

The present invention therefore also has as its object a liquid or non-solid composition for use in the treatment of nail diseases and/or for promoting nail growth comprising one or more biodegradable or non-biodegradable polymers with prolonged release, and optionally one or more active substances, characterized in that it is in the form of a device intended to be administered by insertion into the empty or prepared spaces between the bed and the nail plate and in that it is optionally used simultaneously or spread out over time with administration by subcutaneous at a distance greater than 1 cm from the edge of the nail, preferably in the finger, except for the last phalanx of one or more solid compositions as described above and comprising one or more biodegradable polymers.

In other words, the present invention therefore also has as its object a liquid or non-solid composition comprising one or more monomers or polymers, biodegradable or not, with prolonged release and possibly one or more antifungals for its use in the treatment of nail diseases by administration by insertion into the empty or prepared spaces between the bed and the nail plate, optionally simultaneously or spread over time with subcutaneous administration at a distance greater than 1 cm from the edge of the nail on the side of its proximal end preferably in the finger with the exception of the last phalanx of one or more solid compositions as described above and comprising one or more biodegradable polymers.

Polymers for non-solid biodegradable compositions that can be used by administration by insertion into the space between the bed and the nail plate can be selected, for example, from the polymers described for example in the international patent application No. WO 2008/049631.

These polymers may thus preferably be chosen from very low molecular weight biodegradable polymers optionally capped at one end with an alkyl group containing from 5 to 18 carbon atoms instead of its carboxylic end selected from polylactic acids, polyglycolic acids, poly(lactide-co-glycolide) acids, and mixtures thereof.

These non-solid and non-solidifiable compositions thus comprising one or more polymers, which are biodegradable with prolonged release, may use the same active substances as these solid compositions of the invention which contain a polymer preferably in PLGA-based forms.

The molecular weight of these polymers or copolymers for non-solid compositions, also called semi-solid, biodegradable, is preferably between 700 and 3000 Daltons, preferably between 800 and 2000 Daltons.

The present invention has in particular the object of a composition comprising one or more non-solid biodegradable polymers as described above, characterized in that the non-solid polymer with prolonged release is chosen from polylactic acids, polyglycolic acids, poly(lactide-co-glycolide)s acids, and mixtures thereof, and in that the molecular weight of these polymers or copolymers is preferably between 700 and 3000 Daltons, preferably between 800 and 2000 Daltons.

The present invention also concerns a nail varnish with antimycotic action comprising one or more antifungals preferably chosen from Nystatin, Amphotericin B, Abafungin, Benzalkonium chloride, Caspofungin, Cetrimide, Clioquinol, Copper sulphate, Haloprogin, Echinocandins, Flucytosine, Mycobactovir, Novexatin, Natamycin, Cetylpyridium chloride, Benzylamine butenafine, Benzoxaboroles (cyclic derivatives of boronic acids), Albaconazole, Arasertaconazole, Bifonazole, Butoconazole, Clotrimazole, Eberconazole, Econazole, Eficonazole, Fenticonazole, Fluconazole, Fosravuconazole, Isoconazole, Irtemazole, Isavuconazole, Ketoconazole, piroctone olamine, climbazole, Liarozole, Lianozole, Luliconazole, Miconazole, Oxadiazole, Oxiconazole, Posaconazole, Pramiconazole, Ravuconazole, Sertaconazole, Sulconazole, Terconazole, Thiazole, Thiabendazole, Oteseconazole, Thiadiazole, Thiamazole, Tioconazole, Voriconazole, Amiodarone, Naftifine, Tavaborole, Butenafine, Flucytosine, Griseofulvin, Caspofungin, Micafungin, Nitric Oxide, Sodium Oxychlorosene, Povidoneiodine, Thiocarbonate tolnafate, Sulbentine, Zync Pyrithione and more particularly among Terbinafine, Terbinafine hydrochloride, Ciclopirox, Ciclopiroxolamine, Itraconazole, and Amorolfin for its use in the treatment of nail diseases by application to the nails simultaneously or spread out over time with subcutaneous administration at a distance greater than 1 cm from the edge of the nail, preferably in the finger, with the exception of the last phalanx of one or more solid compositions comprising one or more biodegradable polymers as described hereinabove and/or with the administration by insertion into the space between the bed and the nail plate of one or more non-solid compositions comprising one or more biodegradable polymers as described above.

As indicated above, the varnishes used contain one or more antifungals which can be chosen from the above mentioned antifungals. These varnishes contain a maximum of 10% therapeutic agent except for Pfizer Trosyd® which may contain up to 28% active substance.

Valeant Pharmaceuticals' Penlac® varnish (ciclopirox solution, 8%), which has been approved by the FDA, can be used for example.

The present invention also has as its object an antifungal pharmaceutical composition comprising one or more antifungals preferably selected from Nystatin, Amphotericin B, Abafungin, Benzalkonium chloride, Caspofungin, Cetrimide, Clioquinol, Copper sulphate, Haloprogine, Echinocandins, Flucytosine, Mycobactovir, Novexatin, Natamycin, Cetylpyridium chloride, Benzylamine butenafine, Benzoxaboroles (cyclic boronic acid derivatives), Albaconazole, Arasertaconazole, Bifonazole, Butoconazole, Clotrimazole, Eberconazole, Econazole, Eficonazole, Fenticonazole, Fluconazole, Oteseconazole, Fosravuconazole, Isoconazole, Irtemazole, Isavuconazole, Ketoconazole, Liarozole, Lianozole, Luliconazole, Miconazole, Oxadiazole, Oxiconazole, Piroctone olamine, Climbazole, Posaconazole, Pramiconazole, Ravuconazole, Sertaconazole, Sulconazole, Terconazole, Thiazole, Thiabendazole, Thiadiazole, Thiamazole, Tioconazole, Voriconazole, Amiodarone, Naftifine, Tavaborole, Butenafine, Flucytosine, Griseofulvin, Caspofungin, Micafungin, Viabecline, Nitric Oxide, Sodium Oxychlorosene, Povidoneiodine, Thiocarbonate Tolnafate, Sulbentine, Zync Pyrithione and more particularly among Terbinafine, Ciclopirox, Ciclopiroxolamine, Itraconazole, and Amorolfin for its use in the treatment of nail diseases by oral or injectable administration simultaneously or spread out in time with subcutaneous administration at a distance greater than 1 cm from the edge of the nail on the side of its proximal end preferably in the finger with the exception of the last phalanx of one or more solid compositions comprising one or more biodegradable polymers as described hereafterabove and/or with the administration by insertion into the space between the bed and the nail plate of one or more non-solid polymeric compositions comprising one or more biodegradable as described above and/or with the application to the nails of a nail varnish with an antifungal action as described above.

The invention also relates to a nail varnish with antimycotic action comprising one or more antifungals preferably selected from fluconazole, ketoconazole, miconazole, tioconazole, tavaborole, terbinafine, terbinafine hydrochloride, tolnaftate, amorolfine, ciclopirox, ciclopiroxolamine, terbinafine, terbinafine hydrochloride, or miconazole, for its use in the treatment of nail diseases and/or for promoting nail growth by application to the nails simultaneously or spread out in time with the administration in the finger subcutaneously at a distance greater than 1 cm from the edge of the nail on the side of its proximal end preferably with the exception of the last phalanx of one or more compositions and/or with the administration by insertion into the space between the bed and the nail plate of one or more solid, liquid or non-solid compositions as described above.

Oral antifungals may be selected from the above list, preferably ketoconazole, itraconazole, terbinafine hydrochloride, fluconazole, griseofulvin and in case of hospital emergency treatment posaconazole or voriconazole.

As indicated above, treatments using varnishes and/or oral treatments have disadvantages to which the administration by subcutaneous route at a distance greater than 1 cm from the edge of the nail on the side of its proximal end preferably in the finger with the exception of the last phalanx of one or more compositions comprising one or more biodegradable polymers as described above provide very advantageous therapeutic solutions.

It may however, in certain cases, be interesting to combine these treatments by varnish and/or oral route, for a limited period of time and with possibly reduced doses, with administration by subcutaneous route at a distance greater than 1 cm from the edge of the nail on the side of its proximal end preferably in the finger, with the exception of the last phalanx of one or more compositions comprising one or more solid biodegradable polymers as described above.

These different treatments can thus be combined in order to reinforce (boost) their effectiveness. The use of compositions comprising one or more solid biodegradable sustained-release polymers, and possibly one or more active substances, can thus be added, for example, to an oral treatment as described above.

The invention therefore also has as its object a composition for its use in the treatment of nail fungus, characterized in that it comprises one or more polymers selected from PLA and PGLA, between 0.04 and 6.5 mg of terbinafine hydrochloride, terbinafine, ciclopirox or ciclopiroxolamine, itraconazole and in that it is in the form of an implant with a length of between 4 and 15 mm and a diameter of between 0.3 and 0.8 mm intended to be administered subcutaneously at a distance of more than 1 cm from the edge of the nail on the side of its proximal end in the finger with the exception of the last phalanx.

The present invention also concerns a composition as described above, characterized in that it is preferably in the form of an implant, optionally with a sharpened end, a cylindrical tube or a thread, and in that the composition is administered at a distance of more than 1 cm from the edge of the nail at its proximal end, preferably in the finger, with the exception of the last phalanx, for use in the treatment of nail diseases by simultaneous or staggered administration over time:
the administration by insertion into the space between the bed and the nail plate of one or more solid compositions according to the invention comprising one or more sustained-release biodegradable polymers, and optionally one or more active substances, preferably one or more antifungal agents and/or
the administration by insertion into the space between the bed and the nail plate of a non-solid composition comprising one or more sustained-release biodegradable polymers, and optionally one or more active substances, preferably one or more antifungal agents,
the application to the nails of a nail varnish with antimycotic action comprising one or more antifungals preferably selected from fluconazole, ketoconazole, miconazole, terbinafine, terbinafine hydrochloride, tolnaftate, amorolfine, ciclopirox, ciclopiroxolamine or miconazole, and/or
the oral or injectable administration of an antifungal pharmaceutical composition comprising one or more antifungals preferably selected from azole derivatives such as ketoconazole, fluconazole, isavuconazole, itraconazole, posaconazole or voriconazole; echinocandins such as anidulafungin, caspofungin, micafungin, flucytosin, griseofulvin and terbinafine; amphotericin B and/or
a LASER treatment causing the destruction of the fungus by photobiological thermal effect.

The invention also has as its object, a composition as defined above comprising one or more solid polymers, biodegradable or not, with prolonged release, and optionally one or more active substances, characterized in that it is preferably in the form of an implant optionally having a sharpened end, of a cylindrical tube or a thread and that the composition is administered in the finger at a distance of more than 1 cm from the edge of the nail at its proximal end, with the exception of the last phalanx for its use in the treatment of nail diseases by simultaneous or staggered administration with:
the administration by insertion into the space between the bed and the nail plate of a solid composition comprising one or more sustained-release biodegradable polymers, and optionally one or more active substances, preferably one or more antifungal agents and/or
the administration by insertion into the space between the bed and the nail plate of a liquid or non-solid composition comprising one or more sustained-release biodegradable polymers, and optionally one or more active substances, preferably one or more antifungal agents and/or
the application to the nails of a nail varnish with antimycotic action comprising one or more antifungals preferably selected from fluconazole, ketoconazole, miconazole, terbinafine, terbinafine hydrochloride, tolnaftate, amorolfine, ciclopirox, ciclopiroxolamine, tioconazole or miconazole, and/or
the oral or injectable administration of an antifungal pharmaceutical composition comprising one or more antifungals preferably selected from azole derivatives such as ketoconazole, fluconazole, isavuconazole, traconazole, posaconazole or voriconazole; echinocandins such as anidulafungin, caspofungin, micafungin, flucytosine, griseofulvin and terbinafine; amphotericin B and/or
a laser treatment causing the destruction of the fungus by photobiological thermal effect.

The present invention also concerns a composition as described above, characterized in that it is preferably in the form of an implant, optionally with a sharpened end, a cylindrical tube or a thread and in that the composition is administered in the finger at a distance of more than 1 cm from the edge of the nail towards its proximal end, with the exception of the last phalanx, for its use in the treatment of nail diseases by simultaneous or staggered administration over time:
the administration by insertion into the space between the bed and the nail plate of a non-solid composition comprising one or more sustained-release biodegradable polymers, and optionally one or more active substances, preferably one or more antifungal agents and/or
the application to the nails of a nail varnish with antimycotic action comprising one or more antifungals preferably selected from fluconazole, ketoconazole, miconazole, terbinafine, tolnaftate, amorolfine, ciclopirox, terbinafine hydrochloride, ciclopiroxolamine, tioconazole or miconazole, and/or
the oral or injectable administration of an antifungal pharmaceutical composition comprising one or more antifungals preferably selected from azole derivatives such as ketoconazole, fluconazole, isavuconazole, itraconazole, posaconazole or voriconazole; echinocandins such as anidulafungin, caspofungin, micafungin, flucytosin, griseofulvin and terbinafine; amphotericin B and/or
a LASER treatment causing the destruction of the fungus by photobiological thermal effect.

The use of a LASER beam for the purpose of destroying fungi is known to the man skilled in the art and is described above.

The applicant has also discovered manufacturing conditions, using an advantageous process, which allows some of the said compositions to be obtained according to the invention. Indeed, one of the constraints of the local-regional administration of the compositions according to the invention is that the area of administration is a restricted space and therefore strongly limits the quantity of formulation that can be administered. Furthermore, it is therefore very important to be able to administer formulations with a high content of active substances, which is antinomic with the need to add to the active substances pharmaceutical ingredients that help control the slow release of the active substances in sufficient quantities for a long-lasting action, and thus avoid having to repeat the administrations too often, less than a month or even a week.

It is also obvious to the person skilled in the art that the higher the content of active substance (or core contenting) in sustained-release pharmaceutical compositions, the more difficult it is to obtain a controlled release and therefore a prolonged duration of action.

Surprisingly and unexpectedly, a solution found in our new composition is to associate the active substance in the form of particles in large quantities and to keep them associated but isolated in the preparation process by a small quantity of polymer which will be able to cover the particles to isolate them even with a low percentage of this polymer in the mixture.

It has been observed that under certain manufacturing conditions, it was possible to obtain such a formulation in the solid form of an implant consisting of the said particles of substances surrounded by polymer whose surface appearance is therefore rough, stringy or unsmooth and whose cross section reveals this assembly of the active substance in the form of transparent, crystalline or vitreous particles surrounded by opaque, white, stringy-looking polymer.

Surprisingly and unexpectedly, this assembly provides a controlled and prolonged delay effect, for example over several months as the active substance particles are exposed to the biological tissues or the surrounding environment at the administration site so that the product can then be released when the polymer is about to bioresorb.

In addition, the non-smooth biocompatible and bioresorbable surface of the resulting compositions allows for good adhesion to the subcutaneous tissue or the surrounding environment. The irregularities thus multiply the surface area in contact with the tissue during implantation or insertion and thus ensure locoregional retention in the desired location.

The compositions according to the invention may be prepared by techniques known to person skilled in the art for obtaining sterile parenteral pharmaceutical forms containing biodegradable polymers.

Preferably, without this being limiting, the various constituents of the compositions can be, after weighing, sieving and mixing for non-solid forms, subjected to an extrusion-melting, injection moulding, 3D printing or compacting process to obtain the final shape of the solid forms with the desired geometry and prolonged effect.

Preferably, in a melt-extrusion process the temperature will be advantageously chosen to melt the polymer or polymer mixture around the solid, unmelted active substances, thus allowing partial or total coverage of the active substance to achieve the desired prolonged release effect. This melting operation takes place at the same time as the extrusion operation, with or without stretching to allow this spinning of the various components.

The choice of the operating conditions associated with the composition makes it possible to control the internal and external organisation of the various components and thus ensure its activity.

Surprisingly, without any pretreatment operation with solvents or drying of the solid particles before mixing, mixtures of compositions according to the invention make it possible to obtain homogeneous compositions.

Depending on the grade and the polymer manufacturer, it is supplied in solid form as granulated pellets, “filaments”, or ready-to-use powders, obtained in most cases by cryo-grinding the polymers.

If the polymer is not supplied in powder form for direct mixing with the active substance(s) and other components, an operation designed to reduce their bulk density and facilitate the mixing of the powders before shaping can be advantageously applied.

As a non-limiting example, the person skilled in the art can choose to cryo-grind the polymer, to compacting it and dry granulating it at a temperature lower or equal to 25° C., or even proceed with granulation in an organic solvent or an extrusion-grinding step.

Once the polymer has been reduced to powder form and mixed or not with the other components, the mixture is introduced into the extruder cavity where it is then brought to its melting temperature directly and rapidly during the extrusion process.

In the same way, if one of the ingredients is not directly available in solid form, it will be reduced by applying one of the techniques known to the person skilled in the art for its galenic form only.

The mixture of the active substances with the polymer or copolymer of a lactic acid and/or glycolic acid or the mixture of polymers and/or copolymers of lactic acid and/or glycolic acid forms a melted mixture paste in the presence of the active substances.

The said mixture is thus fed into an extrusion screw according to a process such that the liquefaction-melting time of the mixture and transit time to the extrusion nozzle is reduced to less than 30 minutes and preferably less than 15 minutes.

With this advantageous variant of the production process, it is possible to operate without pre-treatment of the mixture, i.e. without aqueous or organic solvents and/or without freeze-drying of the mixtures and without separate preheating for compression before extrusion. This makes it possible to control the low hydration state of the mixture, if necessary, and to extrude at temperatures of up to 100° C., preferably between 110 and 185° C., and more preferably between 125° C. and 175° C. or between 137 and 173° C., e.g. at approximatively 170° C., without degradation of the active substance, over short heating times of less than 15 minutes, preferably between 5 and 10 minutes.

In the case of extrusion, this hot arrangement can give the desired shape directly by mixing the screw and the diameter of the extrusion nozzle. It will also be possible to control the solid shape and in particular its diameter by means of a stretcher regulating the diameter of the extrudate.

In this case and depending on the desired diameter, the stretcher can operate at room temperature at the extruder outlet. The extrudate may also pass through a chamber thermostatically controlled at high temperature, equal or lower than the extrusion temperature to allow for greater drawing and in particular to obtain very small diameters (for example less than 0.1 mm or even less than 0.05 mm).

This continuous extrudate can then be cut to the size (exchange surface) offering the desired release profile, e.g. by cryo-grinding or by means of a guillotine at the extrusion outlet.

Depending on the shape, the dose and the desired release profile, this manufacturing process can also be applied to shapes with low contents of active substance, less than 20%, in particular between 0.1 and 10%, or high contents, greater than 50%, in particular between 60 and 80%.

The implant can then be placed in an injection device and gamma-irradiated before administration.

The injection device can be pre-filled and already contain the implant, or can be contented just before administration. Similarly, when it is necessary to inject several implants per finger/toe or per person, it may be advantageous in terms of packaging and cost effectiveness to use a reusable device. In this case, the implant is contained directly in the administration needle, which can be changed for each administration.

If the device is not designed to be reused, it can be fitted with a locking system to prevent it from being used again. This system can also hide the needle from the user's view to reduce the risk of possible needlestick injuries.

In another mode of carrying out the invention, the present application has as its object a method of treating diseases of the nail comprising the use of injectable compositions with a prolonged duration of action, more particularly one or more solid biodegradable polymers with prolonged release, and optionally one or more active substances, and in that it is in the form of a device intended to be administered subcutaneously at a distance greater than 1 cm from the edge of the nail on the side of its proximal end preferably in the finger with the exception of the last phalanx.

### EXPERIMENTAL PART

### I—General Procedures

### A—General Procedure for the Preparation of the Composition of the Examples

The compositions according to the invention presented in the examples below are obtained from pharmaceutical processes known to person skilled in the art.

Depending on the use made of the different compositions, batch sizes can vary from a few milligrams to several hundred grams or even kilograms.

The biodegradable polymer, mixture of polymers or (co)polymers used in the following examples are advantageously sieved beforehand on a 600 micrometer mesh or crushed in a cryogenic grinder (cyogenic grinder) type SPEX 6775 Freezer/Mill®.

At the end of the protocol, a fine and homogeneous powder is recovered and can be used in the different preparation procedures.

Subsequently, the polymer or (co)polymer mixture, the active substance, the different excipients or mixtures of active substances and excipients are weighed separately.

The components are then mixed using a Turbula® Type T 2F upside-down mixer (stirrer or stirrer) at a speed of 49 rpm for 10 minutes.

The percentages (%) in the following examples are expressed in mass (m/m).

(1) Extrusion

After mixing the powders making up the compositions, the mixture is then subjected to a continuous melt-extrusion process on a single-screw extruder equipped with an 8 mm pitch screw, pressure control, a variable diameter die or nozzle and a continuous stretching system at the die exit to regulate the diameter of the implants.

The temperature during this process is adapted according to the compositions and active substances and is detailed in each example to be followed. Stretching can also be regulated according to the desired final diameter.

After chemical analysis of the active substance content and purity, the implant is cut to the desired length and sterilised by gamma irradiation at 25 kGy.

(2) Compression

In this manufacturing process, in order to reduce the apparent volume of the polymer used, a first step of dry granulation is carried out. The components are then mixed and used to feed the hopper of the compression machine.

The polymer or (co)polymer mixture, the active substance or mixtures of active substances are compressed using a press, e.g. a Korsch PH106DMS rotary tabletting machine.

Depending on the size and shape of the punch used during compression, it is possible to obtain implants of varying dimensions, active substance content and mass.

The resulting implants are then sterilised by gamma-irradiation at 25 kGy

(3) 3D Printing

The extrudate obtained by extrusion according to the process described above in (1) can be flexible and therefore wound as a yarn or filament. It is possible to manufacture an extrudate with a diameter of 1.75 mm for use with a 3D printer of the FDP (Fusion Deposition Modeling) type, such as the PRUSA i3 HD, with the following characteristics:
Filament diameter: 1.75 mm
Extruded layer height: 0.10 mm
Extruder hotend pack V5: PEEK-brass nozzle, 0.35 mm diameter
Electronics: ARDUINO MEGA 2560* RAMPS v1.4
Platform: glass bed with temperature control

Once printed and cooled, the implants are retrieved with a clamp on the glass platform before analysis. The resulting implants are then sterilised by gamma-irradiation at 25 kGy

### B—General Procedure for the Analysis of Implants

In the procedures set out below in B (1) to (6), the analyses are carried out on a reverse-phase high-performance liquid chromatograph (RP-HPLC), such as the Waters Alliance systems. Detection is performed by a UV/Visible detector, such as Waters' UV/Visible detector 26952489 at a wavelength that varies according to the active substance.

The chromatograph is equipped with a Waters X Bridge C18, 20×4.6 mm, 5 μm type pre-column and a Waters X Bridge C18, 150×4.6 mm, 5 μm type column. The column temperature is maintained at 35° C.

(1) RP-HPLC Method to Determine Purity, Identify and Quantify Terbinafine

### Sample Preparation

Blank: Acetonitrile/Water Milli-Q (25/75, v/v)

The standards are prepared at a concentration of 30 μg/mL terbinafine in a MilliQ (25/75, v/v) acetonitrile/water solution.

The test samples are solubilised and then diluted to approximately 30 μg/mL terbinafine in an acetonitrile/Milli-Q (25/75, v/v) water solution.

### HPLC Chromatography Conditions

An elution gradient is applied from 2 mobile phases:
Mobile phase A: THF/ACN/Citrate Buffer pH4.5 (10/20/70) (v/v/v)
Mobile phase B: Acetonitrile

| t (min) | A (%) | B (%) |
|---|---|---|
| 0 | 80.0 | 20.0 |
| 25.0 | 55.0 | 45.0 |
| 26.0 | 80.0 | 20.0 |
| 31.0 | 80.0 | 20.0 |

Flow: 0.9 mL/min
Injection volume: 30 μL
Analysis time: 32 min.

The detection of the main terbinafine peak is done at the wavelength of 280 nm/226 nm. To quantify and evaluate purity, only the wavelength of 280 nm is used.

Results for active content and purity are determined from the chromatogram of the reference standard and the chromatogram of terbinafine at an approximate retention time of 13.5 min

(2) Simplified Method for the Quantification of Terbinafine

### Test In Vitro

In order to evaluate the release properties of the active substance contained in the implant, an In Vitro release test is carried out at 37° C. for 7 days and then the temperature is raised to 55° C. after 7 days in a physiologically acceptable medium (0.9% NaCl). A minimum of 3 individual samples from the same batch is tested.

The pre-weighed implants are introduced directly into tanks containing 5 mL of physiological medium. 100 μL of the solution are extracted at predetermined times and stored at room temperature until analysis.

The predetermined times are: t=0, t=1 h, t=5 h, t=1 j, t=2 j, t=4 j, t=7 j, t=8 j, t=9 j and t=15 j.

### Sample Preparation

The samples are directly analysed or can be diluted a priori depending on the concentration (dilution with acetonitrile solution/water Milli-Q (25:75, v/v)).

Blank: acetonitrile/Milli-Q water (25:75, v/v)

The standards are prepared at a concentration of 30 μg/mL terbinafine in a Milli-Q (25:75, v/v) acetonitrile/water mixture.

### HPLC Chromatography Conditions

An elution gradient is applied from 2 mobile phases:
Mobile phase A: THF/ACN/Citrate Buffer pH4.5 (10/20/70) (v/v/v)
Mobile phase B: Acetonitrile/Water Milli-Q (95/5, v/v)

| t (min) | A (%) | B (%) |
|---|---|---|
| 0 | 80.0 | 20.0 |
| 4.0 | 10.0 | 90.0 |
| 6.0 | 80.0 | 20.0 |
| 10.0 | 80.0 | 20.0 |

Flow: 0.9 mL/min
Volume injected: 30 μL
Analysis time: 10 min.

The detection of the main terbinafine peak is at the wavelength of 280 nm.

The results for active substance content and purity are determined from the chromatogram of the reference standard and the terbinafine chromatogram at a retention time of approximately 6.4 min.

(3) Method for Determining Purity, Identifying and Quantifying Ciclopirox

### Sample Preparation

Blank: Acetonitrile/water Milli-Q (15/85, v/v)

The standards are prepared at a concentration of 25 μg/mL ciclopirox in a Milli-Q (15/85, v/v) acetonitrile/water solution.

Samples are diluted to approximately 25 μg/mL ciclopirox in Milli-Q (15/85, v/v) acetonitrile/water solution.

Samples, blanks and standards are prepared according to the following process (before injection into the HPLC system):
800 μL sample
400 μL of 0.1M NaOH
80 μL of Dimethyl Sulphate
Vortex Mixing (Agitator) (15 seconds)
Incubation at 37° C. (15 minutes)
80 μL Triethylamine
Vortex Mixing (15 seconds)

### Conditions HPLC Chromatography

An elution gradient is applied from 2 mobile phases:
Mobile phase A: Milli-Q water
Mobile phase B: Acetonitrile

| t (min) | A (%) | B (%) |
|---|---|---|
| 0 | 85 | 15 |
| 25 | 50 | 50 |
| 26 | 20 | 80 |
| 27 | 85 | 15 |
| 32 | 85 | 15 |

Flow: 1.0 mL/min
Volume injected: 60 μL
Analysis time: 32 minutes

The main peak of ciclopirox is detected at the wavelength of: UV @ 305 nm.

Results for active content and purity are determined from the chromatogram of the reference standard and the chromatogram of the ciclopirox at a retention time of approximately 20.2 min.

(4) Simplified Method for the Quantification of Ciclopirox

### Test In Vitro

In order to evaluate the release properties of the active substance contained in the implant, an In Vitro release test is carried out at 37° C. for 7 days in a physiologically acceptable medium (PBS pH 7.4). A minimum of 3 individual samples from the same batch is tested.

The pre-weighed implants are introduced directly into tanks containing 15 mL of physiological medium. 100 μL of the solution are extracted at pre-determined times and stored at 5° C. until analysed.

The pre-determined times are: t=0, t=1 h, t=5 h, t=1 d, t=2 d, t=4 d and t=7 d.

### Sample Preparation

Blank: Acetonitrile/water Milli-Q (15/85, v/v)

The standards are prepared at a concentration of 25 μg/mL ciclopirox in a Milli-Q (15/85, v/v) acetonitrile/water solution.

The samples are injected directly from the In Vitro tests.

Samples, blanks and standards are prepared according to the following process (before injection into the HPLC system):
800 μL sample
400 μL of 0.1M NaOH
80 μL of Dimethyl Sulphate
Vortex mixing (about 15 seconds)
Vortex Mixing (15 seconds)
80 μL Triethylamine
Vortex Mixing (15 seconds)

### Conditions HPLC Chromatography

An elution gradient is applied from 2 mobile phases:
Mobile phase A: Milli-Q water
Mobile phase B: Acetonitrile

| T (min) | A (%) | B (%) |
|---|---|---|
| 0 | 85 | 15 |
| 4 | 0 | 100 |
| 5 | 0 | 100 |
| 6 | 85 | 15 |
| 9 | 85 | 15 |

Flow: 1.0 ml/min
Volume injected: 60 μL
Analysis time: 9 min

The main peak of ciclopirox is detected at the wavelength of 305 nm.

Results for active content and purity are determined from the chromatogram of the reference standard and the chromatogram of the ciclopirox at a retention time of approximately 5.3 min

(5) RP-HPLC Method to Determine Purity, Identify and Quantify Amorolfine

### Test In Vitro

In order to evaluate the release properties of the active substance contained in the implant, an In Vitro release test is carried out at 37° C. for 7 days and is increased to 55° C. after 7 days in a physiologically acceptable medium (0.9% NaCl). A minimum of 3 individual samples from the same batch is tested.

The pre-weighed implants are introduced directly into tanks containing 5 mL of physiological medium. 100 μL of the solution are extracted at predetermined times and stored at room temperature until analysis. The medium is not refilled after volume extraction.

The predetermined times are: t=0, t=1 h, t=5 h, t=1 j, t=2 j, t=4 j, t=7 j, t=8 j, t=9 j and t=15 j.

### Sample Preparation

Blank: Acetonitrile/water Milli-Q (25/75, v/v)

The standards are prepared at a concentration of 200 μg/mL amorolfine in a Milli-Q (25/75, v/v) acetonitrile/water solution.

Samples are diluted to approximately 200 μg/mL amorolfine in Milli-Q (25/75, v/v) acetonitrile/water solution.

### HPLC Chromatography Conditions

An elution gradient is applied from 2 mobile phases:
Mobile phase A: THF/ACN/Citrate Buffer pH4.5 (10/20/70) (v/v/v)
Mobile phase B: Acetonitrile

| t (min) | A (%) | B (%) |
|---|---|---|
| 0 | 80 | 20 |
| 25 | 55 | 45 |
| 26 | 80 | 20 |
| 31 | 80 | 20 |

Flow: 0.9 ml/min
Volume injected: 30 μL
Analysis time: 32 minutes

The detection of the main amorolphine peak is at a wavelength of 224/265 nm. To quantify and evaluate purity, only the wavelength of 265 nm is used.

Results for active content and purity are determined from the chromatogram of the reference standard and the amorolfin chromatogram at an approximate retention time of 12.6 min.

(6) RP-HPLC Method to Determine Purity, Identify and Quantify Itraconazole

### In Vitro

In order to evaluate the release properties of the active substance contained in the implant, an In Vitro release test is carried out at 37° C. for 7 days and then the temperature is raised to 55° C. after 7 days in a physiologically acceptable medium (0.9% NaCl). A minimum of 3 individual samples from the same batch is tested.

The pre-weighed implants are introduced directly into tanks containing 5 mL of physiological medium. 100 μL of the solution are extracted at predetermined times and stored at room temperature until analysis. The medium is not refilled after volume extraction.

The predetermined times are: t=0, t=1 h, t=5 h, t=1 j, t=2 j, t=4 j, t=7 j, t=8 j, t=9 j and t=15 j.

### Sample Preparation

Blank: THF/ACN/Mobile Phase A (15/10/75) (v/v/v)

The standards are prepared at a concentration of 50 μg/mL itraconazole in a THF/ACN/mobile phase A solution (15/10/75) (v/v/v).

Samples are diluted to an approximate concentration of 50 μg/mL itraconazole with a THF/ACN/mobile phase A (15/10/75) solution (v/v/v).

### HPLC Chromatography Condition

An elution gradient is applied from 2 mobile phases:
Mobile phase A: THF/ACN/Buffer Citrate pH 4.5 (10/20/70) (v/v/v)
Mobile phase B: Acetonitrile

| t (min) | A (%) | B (%) |
|---|---|---|
| 0 | 80 | 20 |
| 25 | 55 | 45 |
| 26 | 80 | 20 |
| 31 | 80 | 20 |

Flux: 0.9 mL/min
Injected volume: 25 μL
Analysis time: 32 min.

The main peak of itraconazole is detected at a wavelength of 265 nm.

The results for active content and purity are determined from the chromatogram of the reference standard and the chromatogram of itraconazole at a retention time of approximately 19.0 min.

### C—Evaluation of the In Vitro Antifungal Activity of a Composition According to the Invention

The antifungal activity over time (or inhibition capacity) of the compositions described in Examples 1, 2, 6, 8, 10, 12, 18 and 19 below has been evaluated using the agar composition diffusion test (inhibition of petri dish growth), performed on seeds of isolated Trichophyton Rubrum strains, Candida Krusei and/or Candida Parapsilosis (human clinical samples of Onychomycosis). The antifungal agent released by the composition in the culture medium more or less inhibits the development of the inoculum; this is then compared with the corresponding MIC.

Prior to the diffusion test, the Minimum Inhibitory Concentration (MIC) is carried out by micro-dilution in a liquid medium following the standard CLSI M38-A2 protocol to determine the sensitivity to the fungal agent used in the Agar test. In this protocol, the MIC of the antifungal agent corresponds to 80% inhibition (concerning the growth control of the fungal strain).

Method: A sample of the composition described in Examples 1, 2, 6, 8, 10, 12, 18 and 19 is placed in the centre of a Petri dish (modified Müller-Hinton) and incubated. The change in growth of the inhibition zone (diameter in mm) is measured at predetermined times (in days).

This experiment is carried out according to the standard CLSI M44-A2 protocol—in a modified Müller Hinton environment.

The plates are incubated in a humidity chamber at two parallel incubation temperatures (27° C. and/or 35° C.).

### D—Stability Procedures

Implants manufactured according to Example 1 and Example 18 gamma-irradiated have been stabilised under ICH conditions of stability at different conditions selected from 5° C., 25° C./60% relative humidity and 40° C./75% relative humidity.

The dose and purity of the active substance are evaluated according to the method described above in B at different reference times.

### E—In Vivo Testing Procedures

Implants manufactured according to Example 1, Example 3 and Example 18 were subcutaneously implanted in sprague-dawley male rats.

Implantation is done with a 1 ml Hypak® syringe (Becton Dickinson) precontented with a 20G Henke Sass Wolf needle (0.9 mm outside diameter, 0.6 mm inside diameter, 16 mm length).

At times corresponding to 7 days, 28 days, 56 days, 91 days and 112 days for the implants described in Example 1, 24 days and 84 days for the implants described in Example 3 and 7 days, 30 days and 84 days for the implants described in Example 18 after implantation of the implants, the implants were extracted from animal subcutaneous tissue and the active substance content analysed by RP HPLC-UV according to the method described above in B (1) to B (4).

### II—Detailed Examples

### Example 1

4 batches of implants containing Terbinafine at different concentrations in PLGA 85:15 are prepared according to the procedure described above in A (1).

The extrusion conditions are as follows:

| | Screw | Extrusion | Extrusion | Nozzle | Extrusion |
|---|---|---|---|---|---|
| | diameter | speed | temperature | diameter | outlet |
| Batches | (mm) | (rpm) | (T° C.) | (mm) | pressure |
| (1) | 8 | 6 | 150-210 | 0.5 | N/A |
| | | | 155-162 | | |
| (2) | 8 | 10 | 150-170 | 0.85 | 470-1200 |
| | | | | | 500-1000 |
| | | | | | (mPa) |
| (3) | 8 | 10-13 | 164 | 0.5 | 90-140 bar |
| (4) | 8 | 10-13 | 166 | 0.5 | 200-250 |
| | | | | | (mPa) |

Implants with a length of 10 mm and a diameter of 0.56 mm are cut out.

The batches are then evaluated analytically according to the method described above in B (1) and/or B (2).

| | | Active | | |
|---|---|---|---|---|
| | Implant | substance | | Dose of active |
| | diameter | content | Active | substance/ |
| Batches | (mm) | (% m/m) | purity % | implant (mg) |
| (1) | 0.56 mm | 4.50 | 98.97 | 0.15 |
| (2) | 0.30 mm | 15.70 | 98.10 | 0.13 |
| (3) | 0.40 | 29.90 | 99.51 | 0.44 |
| (4) | 0.58 | 58.20 | 99.59 | 1.61 |

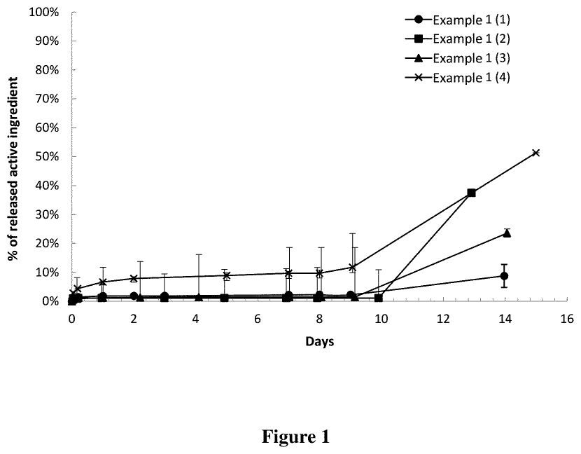

The In Vitro Release Test is performed as described above in B (2). The results are shown in FIG. 1.

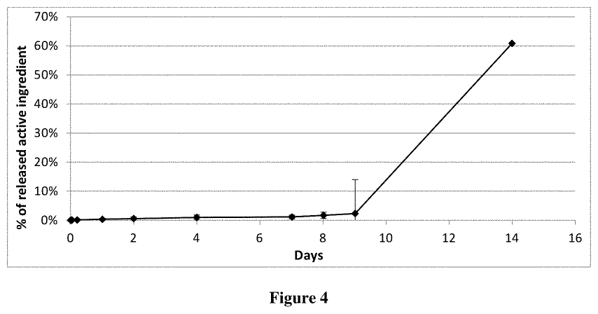

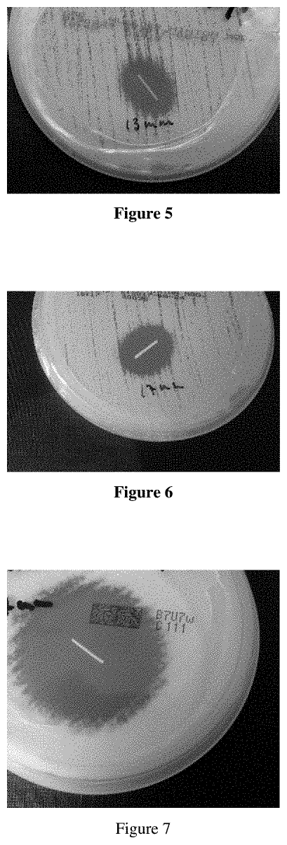

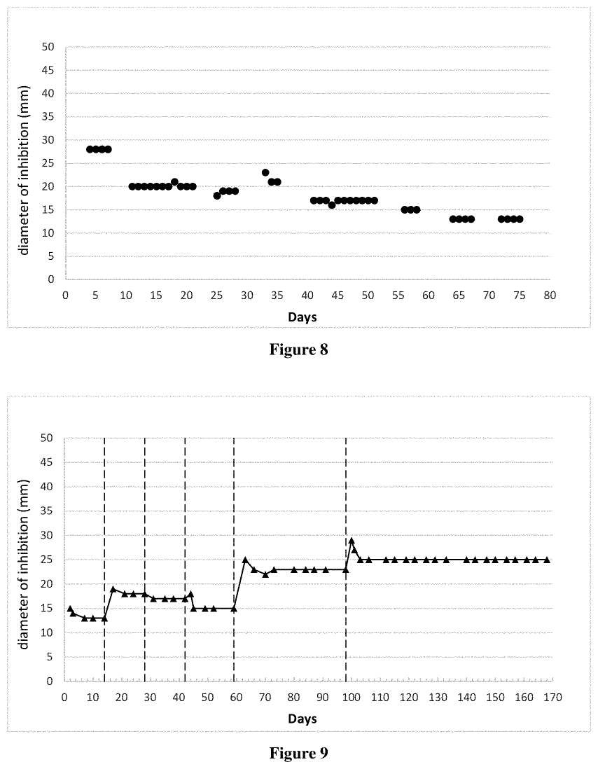

The evaluation of the In Vitro antifungal activity is carried out as described above in C. FIG. 5 shows a picture of an agar plate after implantation of a composition according to Example 1 (1) at 35° C. in C. Parapsilosis.

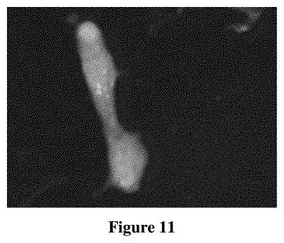

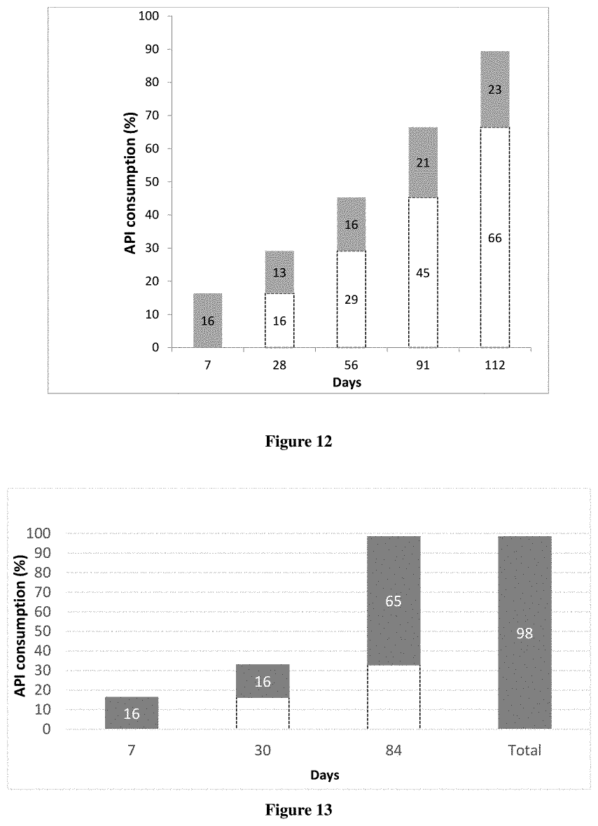

The In Vivo test is performed as described above in E. The In Vivo test results are shown in FIG. 12.

The stability of the batch (3) is determined according to the method described above in D at 5° C., 25° C./60% relative humidity and 40° C./75% relative humidity, at 1 month, 3 months and 6 months. The results of Terbinafine HCl content, purity and dose of active substance obtained are as follows:

| Batche | Parameter | Conditions | T0 | T1 M | T3 M | T6 M |
|---|---|---|---|---|---|---|
| Example | Active | 5° C. | 29.9 | 27.5 | 30.0 | 30.8 |
| 1 (3) | substance | 25° C./ | | 27.9 | 30.3 | 30.8 |
| | content | 60% RH | | | | |
| | (% m/m) | 40° C./ | | 28.9 | NA | NA |
| | | 75% RH | | | | |
| | Purity | 5° C. | 99.5 | 99.4 | 99.4 | 99.4 |
| | (% Ar) | 25° C./ | | 99.5 | 99.4 | 99.4 |
| | | 60% RH | | | | |
| | | 40° C./ | | 99.5 | NA | NA |
| | | 75% RH | | | | |

The stability of the batch (4) is determined according to the method described above in D at 5° C. at 9 months and 12 months. The results of terbinafine content, purity and dose of active substance obtained are as follows:

| | | T0 | T9 M | T12 M |
|---|---|---|---|---|
| Batche | Parameter | 5° C. | 5° C. | 5° C. |
| Example | Content | 58.2 | 58.5 | 57.9 |
| 1 (4) | (%, w/w) | | | |
| | Dose (mg) | 1.61 | 1.58 | 1.54 |
| | Purity | 99.6 | 99.6 | 99.3 |
| | (% Ar) | | | |

### Example 2

A batch of implants containing Terbinafine HCl and PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 3.5-7 | 166 | 0.85 | 2500 |

Implants with a length of 25 mm and a diameter of 0.80 mm are cut out.

| | | Dose of active |
|---|---|---|
| Active substance | Active | substance/ |
| content (% m/m) | purity % | implant (mg) |
| 57.70 | 99.80 | 5.70 |

Implants with a length of 10 mm and a diameter of 0.80 mm are cut out.

| | | Dose of active |
|---|---|---|
| Active substance | Active | substance/ |
| content (% m/m) | purity % | implant (mg) |
| 57.70 | 99.80 | 2.28 |

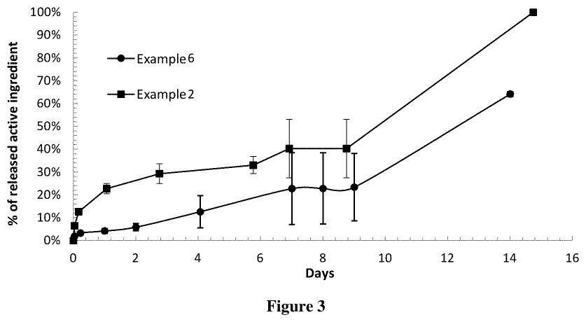

The In Vitro Release Test is performed with 10 mm long implants as described above in B (2). The results are shown in FIG. 3.

The valuation of the In Vitro antifungal activity is carried out according to the method described above in C. FIG. 6 shows a picture of an agar plate after implantation of a compound according to Example 2 in Candidae Parapsilosis. The results are summarised in FIG. 9.

### Example 3

A batch of implants containing dexamethasone and PLGA 75:25 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (bar mPa) |
| 8 | | 130 | 0.85 | 20-40 |

Implants with a length of 6 mm and a diameter of 0.45 mm are cut out.

| | | Dose of active |
|---|---|---|
| Active substance | Active | substance/ |
| content % m/m | purity % % | implant (mg) |
| 55.0 | 99.15 | 0.646 |

The In Vivo test is performed as described above in E.

### Example 4

A batch of implants containing 60% Ciclopiroxolamine and 40% PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 6 | 155 | 0.85 | 2000 |

Implants with a length of 10 mm and a diameter of 0.60 mm are cut out.

### Example 5

A batch of implants containing 50% miconazole and 50% PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 4.5-5.5 | 160 | 0.85 | 1500-200 |

Implants with a length of 9 mm and a diameter of 0.58 mm are cut out.

### Example 6

A batch of implants containing amorolfin and PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 2-10 | 160 | 1.5 | — |

Implants with a length of 10.90 mm and a diameter of 0.58 mm are cut out.

The analyses are carried out according to the procedure described above in B (5).

| Content API % m/m | Active purity % | Dose implant |
|---|---|---|
| 46.30 | 100 | 1.54 |

In Vitro release is performed as described above in B (5). The results are shown in FIG. 3.

The evaluation of the In Vitro antifungal activity is carried out as described above in C.

### Example 7

A batch of implants containing 30% horsetail and 70% PLGA 85:15 is prepared according to the procedure described above in A (1).

The extrusion conditions are as follows:

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 10 at 150° C. 5 min | 150-180 | 0.85 | 2000-3000 |
| | then 2 at 180° C. | | | |

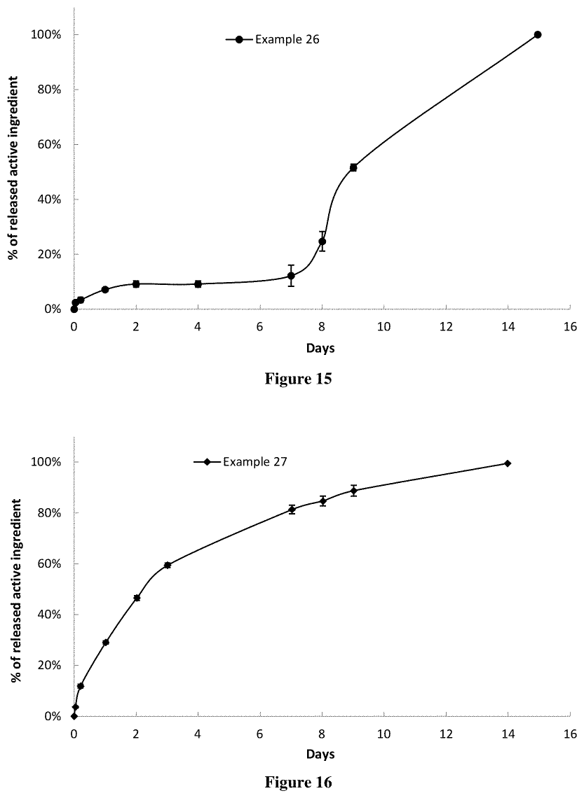

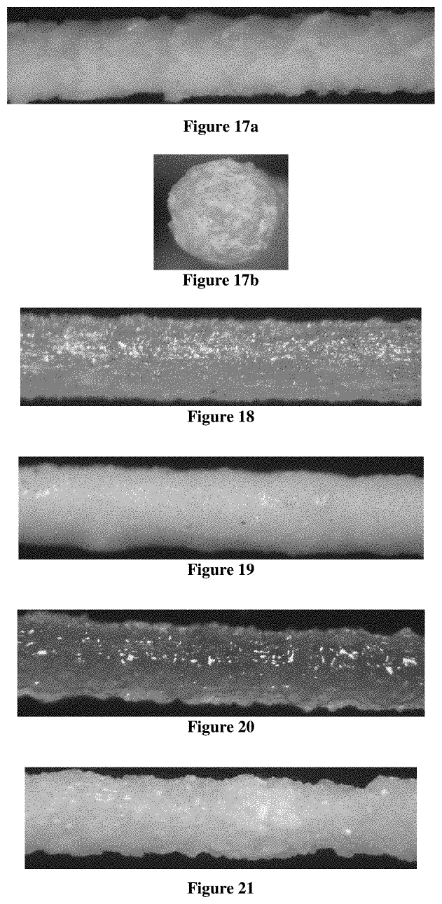

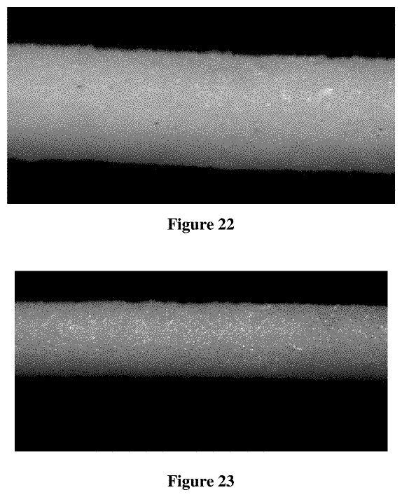

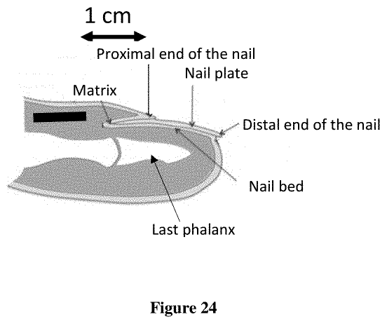

Implants with a length of 10 mm and a diameter of 0.4-0.8 mm are then cut. FIG. 15 shows a photo of the implants in Example 7 taken with a binocular loupe.

### Example 8

A batch of implants containing itraconazole and PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 10 | 110-170 | 0.7 | 3000 |

Implants with a length of 6 mm and a diameter of 0.7 mm are then cut.

The analyses are carried out according to the procedure described above in B (6).

| Content % m/m | Active purity % | Dose implant |
|---|---|---|
| 60.90 | 99.3 | 1.9 |

FIG. 16 shows a photo of the implants in Example 8 taken with a binocular loupe.

### Example 9

A batch of implants containing 12% lactate, PLGA 85:15 and Terbinafine HCl is prepared according to the procedure described in A (1) above.

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 12 | 110-120 | 0.85 | 750 |

Implants with a length of 10 mm and a diameter of 0.8 mm are cut out.

The analyses are carried out according to the procedure described above in B (1) and B (2).

| Content % m/m | Active purity % | Implant dose mg |
|---|---|---|
| 58.20 | 99.53 | 2.15 |

The In Vitro release is made as described above in B (2).

### Example 10

A batch of implants containing 50% PLGA 85:15 and 50% L-lactate is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 10-20 | 120 | 0.85 | 49 |

Implants with a length of 10 mm and a diameter of 0.8 mm are cut out.

### Example 11

A batch of implants containing 12% PLGA 50:50, 28% PLGA 85:15 and 60% Terbinafine HCl is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 10 | 166-100 | 0.5-0.85 | 1000-2000 |

Implants with a length of 10 mm and a diameter of 0.6 mm are cut out.

The analyses are carried out according to the procedure described above in B (1) and B (2).

| Content % m/m | Active purity % | Implant dose mg |
|---|---|---|
| 59.60 | 99.53 | 1.65 |

### Example 12

A batch of implants containing 100% PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 2-3 | 146-154 | 0.85 | — |

Implants with a length of 10 mm and a diameter of 0.80 mm are cut out.

An antifungal activity test is carried out as described above in C with two strains: Candida Krusei and Candida Parapsilosis. The results of the inhibition halo are given in millimetres around the implant in the following table:

| t | 10 days | 14 days | 20 days | 28 days |
|---|---|---|---|---|
| Candida Krusei | 4 mm | — | 3 mm | 3 mm |
| Candida | 4 mm | 5 mm | 3 mm | 3 mm |
| Parapsilosis | | | | |

### Example 13

A batch of implants containing 12% PLA 202S, 28% PLGA 85:15 and 60% Terbinafine HCl is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 8 | 100 | 0.85 | 1600 |

Implants with a length of 10 mm and a diameter of 0.55 mm are cut out.

The analyses are carried out according to the procedure described above in B (1) and B (2).

| Content % m/m | Active purity % | Implant dose mg |
|---|---|---|
| 59.30 | 99.53 | 1.51 |

### Example 14

A batch of implants containing 50% PLA 202S and 50% PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 8 rpm | 100 | 0.85 | 1600 |

Implants with a length of 10 mm and a diameter of 0.55 mm are cut out.

### Example 15

A batch of implants containing 25% chitosan and 75% PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 10 | 150 | 0.85 | 1500 |

Implants with a length of 10 mm and a diameter of 0.60 mm are cut out.

### Example 16

A batch of implants containing 20% chitosan, PLGA 85:15 and Terbinafine HCl is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 10 | 148 | 0.85 | 2000 |

Implants with a length of 10 mm and a diameter of 0.60 mm are cut out.

The analyses are carried out according to the procedure described above in B (1) and B (2).

| Content % m/m | Active purity % | Implant dose mg |
|---|---|---|
| 8.80 | 99.63 | 0.33 |

The In Vitro diffusion test is carried out as described above. The results are shown in FIG. 4.

### Example 17

A batch of implants containing Minoxidil and PLGA 85:15 is prepared according to the procedure described above in A (1).

| | | | | Extrusion |
|---|---|---|---|---|
| Screw | Extrusion | Extrusion | Nozzle | outlet |
| diameter | speed | temperature | diameter | pressure |
| (mm) | (rpm) | (T° C.) | (mm) | (mPa) |
| 8 | 10 | 148 | 0.85 | 2000 |

Implants with a length of 8 mm and a diameter of 0.75 mm are cut out.

### Example 18

Implant batches containing different concentrations of ciclopirox in PLGA 85:15 are prepared according to the procedure described above in A (1).

(1)

| | | Extru- | Extrusion | Nozzle | | |
|---|---|---|---|---|---|---|
| | Screw | sion | temper- | diam- | | Length and |
| | diameter | speed | ature | eter | Pres- | diameter |
| | (mm) | (rpm) | (T° C.) | (mm) | sure | (mm) |
| (1) | 8 | 2 | 130 | 0.85 | N/A | 10 × 0.30 mm |
| (2) | 8 | 10-12 | 102 | 0.85 | 150 bar | 10 × 0.40 mm |
| (3) | 8 | 10 | 110 | 0.70 | 75 bar | 10 × 0.60 mm |
| (4) | 8 | 10 | 130 | 1 | N/A | 9 × 0.65 mm |

The analyses are carried out according to the procedure described above in B (3) and B (4).

| | Content % m/m | Active purity % | Implant dose mg |
|---|---|---|---|
| (1) | 15.50 | 97.60 | 0.15 |
| (2) | 34. | 98.10 | 0.52 |
| (3) | 59.690 | 98.40 | 2.50 |
| (4) | 65.80 | 98.20 | 1.80 |

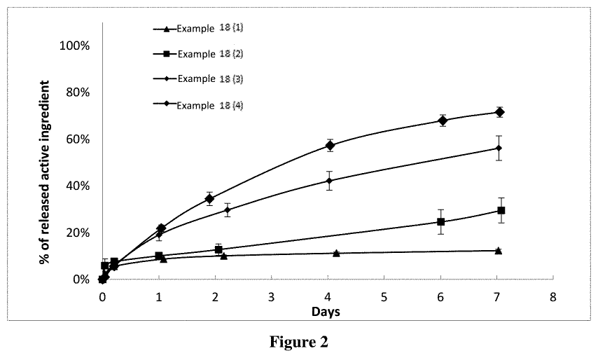

The In Vitro Release Test is performed as described in B (4) above. The results are shown in FIG. 2.

The evaluation of the In Vitro antifungal activity is carried out as described above in C.

The In Vivo test is performed as described above in E.

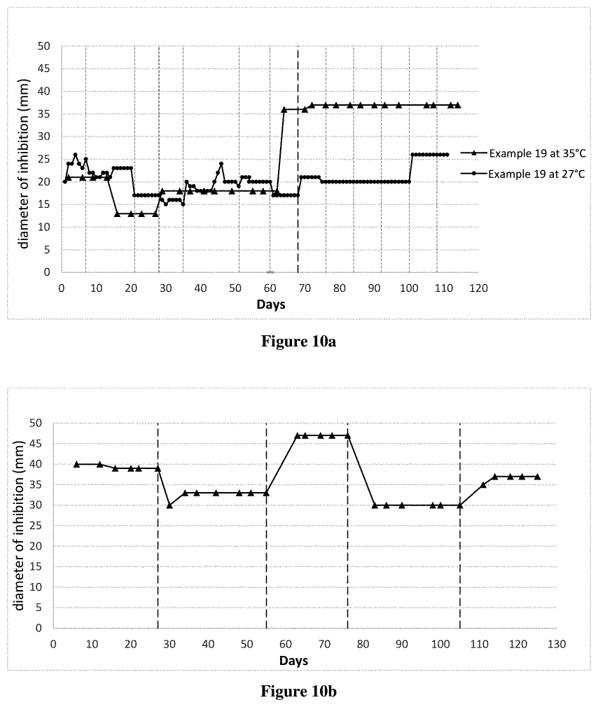

FIG. 11 shows a picture of an implant of the composition described above in 18 (3) after 84 days of implantation.

The results of the In Vivo implementation are summarised in FIG. 13.

The stability of batch (2) is determined as described above in D at 5° C., 25° C./60% relative humidity and 40° C./75% relative humidity, at 1 month, 3 months and 6 months. The results of ciclopirox content, purity and dose of active substance obtained are as follows:

| Batche | Parameter | Conditions | T0 | T1 M | T3 M | T6 M |
|---|---|---|---|---|---|---|
| Example | Active | 5° C. | 34.80 | 33.9 | 35.0. | 36.8 |
| 18 (2) | substance | 25° C./ | | 34.5 | 33.8 | 35.7 |
| | content | 60% RH | | | | |
| | (% m/m) | 40° C./ | | 33.0 | NA | NA |
| | | 75% RH | | | | |
| | Purity | 5° C. | 98.1 | 98.6 | 98.3 | 98.6 |
| | (%, w/w) | 25° C./ | | 98.6 | 98.3 | 98.4 |
| | | 60% RH | | | | |
| | | 40° C./ | | 98.7 | NA | NA |
| | | 75% RH | | | | |
| | Active | 5° C. | 0.51 | 0.46 | 0.52 | 0.54 |
| | dose (mg) | 25° C./ | | 0.53 | 0.49 | 0.51 |
| | | 60% RH | | | | |
| | | 40° C./ | | 0.51 | NA | NA |
| | | 75% RH | | | | |

The stability of the batch (3) is determined according to the procedure described above in D at 5° C., 25° C./60% relative humidity and 40° C./75% relative humidity, at 1 month, 3 months and 6 months. The results of ciclopirox content, purity and dose of active substance obtained are as follows:

| Batche | Parameter | Conditions | T0 | T1 M | T3 M | T6 M |
|---|---|---|---|---|---|---|
| Example | Active | 5° C. | 59.90 | 58.6 | 57.5 | 60.2 |
| 18 (3) | substance | 25° C./ | | 56.8 | 61.3 | 59.4 |
| | content | 60% RH | | | | |
| | (% m/m) | 40° C./ | | 57.6 | NA | NA |
| | | 75% RH | | | | |
| | Purity | 5° C. | 98.40 | 98.4 | 98.4 | 98.8 |
| | (%, w/w) | 25° C./ | | 98.3 | 98.5 | 98.7 |
| | | 60% RH | | | | |
| | | 40° C./ | | 98.3 | NA | NA |
| | | 75% RH | | | | |
| | Active | 5° C. | 2.50 | 2.18 | 2.39 | 2.28 |
| | dose (mg) | 25° C./ | | 2.23 | 2.37 | 2.40 |
| | | 60% RH | | | | |
| | | 40° C./ | | 2.22 | NA | NA |
| | | 75% RH | | | | |

### Example 19

A batch of implants containing Ciclopirox and PLGA 85:15 is prepared according to the procedure described above in A (1).

| Screw | Extrusion | Extrusion | Nozzle | Extrusion |
|---|---|---|---|---|
| diameter | speed | temperature | diameter | outlet |
| (mm) | (rpm) | (T° C.) | (mm) | pressure |
| 8 | 10-17 | 130-126 | 1 | 600-2300 |

Implants with a length of 7 mm and a diameter of 1 mm are cut out.

The analyses are carried out according to the procedure described above in B (3) and B (4).

| Content % m/m | Active purity % | Implant dose mg |
|---|---|---|
| 55.00 | 98.9 | 1.98 |

The evaluation of the In Vitro antifungal activity is carried out according to the procedure described above in C. FIG. 7 shows a picture of an agar plate after implantation of a compound according to Example 19 at 35° C. in C. Parapsilosis.

The results are summarised in FIGS. 10a and 10b.

### Example 20

A mixture of 50% PLGA 85:15 and 50% PLA 202S is prepared according to the process described above in A (2). The punches are placed in a 1.4 mm configuration for a thickness of 4. 19 implants are included in the batch.

The process uses a compression force of 220-250N for an ejection force of 20-25N.

The implants obtained have the following characteristics:

| Mass | Length | Diameter | Density | Volume |
|---|---|---|---|---|
| (mg) | (mm) | (mm) | (g/mL) | (μL) |
| 1.05 | 1.61 | 0.91 | 1.11 | 1.05 |

### Example 21

A filament containing 100% PLGA 85:15 is prepared according to the printing process described above in A (3).

| Screw | Extrusion | Extrusion | Nozzle | Extrusion |
|---|---|---|---|---|
| diameter | speed | temperature | diameter | outlet |
| (mm) | (rpm) | (T° C.) | (mm) | pressure |
| 8 | 12 | 135-141 | 2.5 | 60 MPa |

This 1.55-1.75 mm diameter filament is then used to be printed in 3D as described above in A (3). The printing conditions are as follows:
Extrusion temperature: 190° C.
Glass temperature (bed): 50° C.
Power supply: 60% of the maximum power supply
Speed: 80% of the maximum speed

Implants are printed in batches of 10, and measure 10 mm for 0.4 mm diameter.

### Example 22

A batch of implants containing Ciclopirox and PLA 202H is prepared according to the procedure described above in A (1).

| Screw | Extrusion | Extrusion | Nozzle | Extrusion |
|---|---|---|---|---|
| diameter | speed | temperature | diameter | outlet |
| (mm) | (rpm) | (T° C.) | (mm) | pressure |
| 8 | 6-10 | 80 | 1 | N/A |

Implants with a length of 10 mm and a diameter of 0.8 mm are cut out.

The analyses are carried out according to the procedure described above in B (3) and B (4).

| Content % m/m | Active purity % | Implant dose mg |
|---|---|---|
| 41.90 | 99.95 | 2.37 |

### Example 23

A batch of implants containing approximately 30% (w/w) Arginine and PLGA 85:15 is prepared according to the procedure described above in A (1).

| Screw | Extrusion | Extrusion | Nozzle | Extrusion |
|---|---|---|---|---|
| diameter | speed | temperature | diameter | outlet |
| (mm) | (rpm) | (T° C.) | (mm) | pressure |
| 8 | 8 | 150 | 1 | 1500 MPa |

Implants with a length of 10 mm and a diameter of 0.9 mm are cut out.

### Example 24

A batch of implants containing approximately 30% (w/w) Ciclopirox and 30% (w/w) Terbinafine HCl to obtain in total a mixture with PLGA 85:15 having an active substance content of 60% (w/w) is prepared according to the method described above in A (1).

| Screw | Extrusion | Extrusion | Nozzle | Extrusion |
|---|---|---|---|---|
| diameter | speed | temperature | diameter | outlet |
| (mm) | (rpm) | (T° C.) | (mm) | pressure |
| 8 | 10-12 | 130-140 | 1 | 90-150 bar |

Implants with a length of 10 mm and a diameter of 0.8 mm are cut out.

### Example 25

A batch of implants containing approximately 60% (w/w) Terbinafine-HCl in PLGA 85:15 are prepared according to the procedure described above in A (1).

The extrusion conditions are as follows:

| Screw | Extrusion | Extrusion | Nozzle | Extrusion |
|---|---|---|---|---|
| diameter | speed | temperature | diameter | outlet |
| (mm) | (rpm) | (T° C.) | (mm) | pressure |
| 8 | 10-13 | 166 | 0.5 | 200-250 bar |

Implants with a length of 10 mm and a diameter of 0.56 mm are cut out.

The lot is then evaluated analytically according to the method described above in B (1) and/or B (2).

| | Active | | |
|---|---|---|---|
| Implant | substance | Active | Dose of active |
| diameter | content | purity | substance/ |
| (mm) | (% m/m) | % | implant (mg) |
| 0.56 mm | 58.5 | 99.6 | 1.41 |

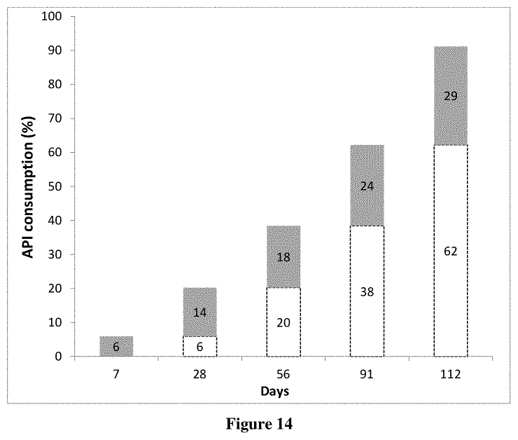

The In Vivo test is performed as described above in E. The In Vivo test results are shown in FIG. 14.

### Example 26

A batch of larger implants (16.1 g or the theoretical equivalent of 6,000 units) containing approximately 60% (w/w) Terbinafine-HCl in PLGA 85:15 are prepared according to the procedure described above in A (1).

The extrusion conditions are as follows:

| Screw | Extrusion | Extrusion | Nozzle | Extrusion |
|---|---|---|---|---|
| diameter | speed | temperature | diameter | outlet |
| (mm) | (rpm) | (T° C.) | (mm) | pressure |
| 8 | 6 | 167 | 0.5 | 230-250 bar |

Implants with a length of 10 mm and a diameter of 0.56 mm are cut out.

The lot is then evaluated analytically according to the method described above in B (1) and/or B (2).

| | Active | | |
|---|---|---|---|
| Implant | substance | Active | Dose of active |
| diameter | content | purity | substance/ |
| (mm) | (% m/m) | % | implant (mg) |
| 0.56 mm | 61.7 | 99.8 | 1.63 |

The In Vitro Release Test is performed as described above in B (2). The results are shown in FIG. 15.

### Example 27

A larger batch of implants (3.9 g or the theoretical equivalent of 1441 units) containing approximately 60% (w/w) Ciclopirox and PLGA 85:15 is prepared according to the procedure described above in A (1).

| Screw | Extrusion | Extrusion | Nozzle | Extrusion |
|---|---|---|---|---|
| diameter | speed | temperature | diameter | outlet |
| (mm) | (rpm) | (T° C.) | (mm) | pressure |
| 8 | 5 | 112 | 0.6 | 107 |

Implants with a length of 10 mm and a diameter of 0.56 mm are cut out.

The analyses are carried out according to the procedure described above in B (3) and B (4).

| Content % m/m | Active purity % | Implant dose mg |
|---|---|---|
| 58.8 | 99.0 | 1.64 |

The In Vitro Release Test is performed as described above in B (2). The results are shown in FIG. 16.

1-19. (canceled)

20. A method for the treatment of nail diseases, comprising: administering a composition comprising one or more solid biodegradable sustained-release polymers, and optionally one or more active substances and wherein the composition is in the form of a device which is administered subcutaneously at a distance greater than 1 cm from the edge of the nail on the side of its proximal end, in the finger with the exception of the last phalanx.

21. The method according to claim 20, comprising one or more active substances selected from:
Anti-infectives selected from:
antifungal agents,
Antibiotics,
and pest control products;
Anti-inflammatory drugs selected from:
steroidal anti-inflammatory drugs,
non-steroidal anti-inflammatory drugs, and
Janus Kinase inhibitors (JAK);
The cyclosporins;
vitamin D or vitamin D derivatives;
Immunosuppressants;
Amino acids;
Active substances that accelerate nail growth selected among:
prostaglandins,
Chitosan, hydroxypropyl chitosan,
Valproic acid,
Echisetum arvense (horsetail), Hyaluronic Acid, Biotin, Growth Hormone (GH),
Minoxidil, Finasteride;
Active substances promoting blood circulation chosen from among vasodilators and
anti-platelet aggregants;
Local anaesthetics.

22. The method according to claim 20, wherein the composition comprises one or more active substances selected from:
Antifungal agents,
Steroidal anti-inflammatory drugs,
Non-steroidal anti-inflammatory drugs and Janus Kinase (JAK) inhibitors,
Active substances accelerating nail growth selected from Prostaglandins, Echisetum arvense (horsetail), Chitosan, Hydroxypropyl chitosan, Hyaluronic acid, Biotin, Valproic acid, Growth hormone (GH), Minoxidil and/or Finasteride,
Amino acids,
Local anaesthetics,
Vasodilators,
Antibiotics,
Pest control products.

23. The method according to claim 20, wherein the nail disease is psoriasis accompanied or not by an inflammatory phenomenon and in that the composition comprises one or more active substances selected from:
Steroidal anti-inflammatory drugs,
Non-steroidal anti-inflammatory drugs,
Janus Kinase inhibitors (JAK),
The cyclosporins,
Amino acids,
Vitamin D or vitamin D derivatives,
immunosuppressants,
retinoids,
compounds such as Anthralin (or dithranol) and Urea,
one or more active substances accelerating nail growth, preferably selected from Prostaglandins, Echisetum arvense (horsetail), Chitosan, Hydroxypropyl chitosan, Hyaluronic Acid, Biotin, Valproic Acid, Growth Hormone (GH), Minoxidil and/or Finasteride,
Vasodilators,
Local anaesthetics.

24. A method for promoting nail growth, comprising a composition comprising one or more solid biodegradable sustained-release polymers and optionally one or more active substances and wherein the composition is in a form of a device which is administered subcutaneously at a distance greater than 1 cm from the edge of the nail on the side of its proximal end, in the finger with the exception of the last phalanx.

25. The method according to claim 24, wherein the composition comprises one or more solid biodegradable sustained-release polymers and one or more active substances selected from:
cyclosporins;
prostaglandins;
Echisetum arvense (horsetail), Chitosan, Hydroxypropyl chitosan, Hyaluronic acid, Arginine, Biotin, Valproic acid, Growth hormone (GH), Minoxidil and/or Finasteride;
Vasodilators;
valproic acid;
and possibly one or more active substances selected from:
anti-infectives selected from:
antifungal agents,
Antibiotics,
and pest control products;
anti-inflammatory drugs selected from
steroidal anti-inflammatory drugs,
non-steroidal anti-inflammatory drugs, and
Janus Kinase inhibitors (JAK);
vitamin D or vitamin D derivatives;
immunosuppressants;
amino acids;
active substances promoting blood circulation chosen from among vasodilators, and
anti-platelet aggregants;
local anaesthetics,
and the composition is in the form of a device intended to be administered subcutaneously at a distance greater than 1 cm from the edge of the nail, in the finger, except for the last phalanx.

26. The method according to claim 24, wherein the method comprises administering a composition comprising one or more solid biodegradable sustained release polymers, one or more active substances selected from:
cyclosporins;
prostaglandins;
Echisetum arvense (horsetail), Chitosan, Hydroxypropyl chitosan, Hyaluronic acid, Arginine, Biotin, Valproic acid, Growth hormone (GH), Minoxidil and/or Finasteride;
Vasodilators;
valproic acid;
and possibly one or more active substances selected from:
anti-infectives selected from:
antifungal agents,
Antibiotics,
and pest control products;
anti-inflammatory drugs selected from
steroidal anti-inflammatory drugs,
non-steroidal anti-inflammatory drugs, and
Janus Kinase inhibitors (JAK);
vitamin D or vitamin D derivatives;
immunosuppressants;
amino acids;
active substances promoting blood circulation chosen from among vasodilators, and
anti-platelet aggregants;
local anaesthetics,
and the composition is in the form of a device intended to be administered subcutaneously at a distance greater than 1 cm from the edge of the nail, in the finger, except for the last phalanx.

27. The method according to claim 20, wherein the composition is in the form of an implant optionally having a sharpened end, a cylindrical tube or a thread and the composition is administered subcutaneously at a distance greater than 1 cm from the edge of the nail on the side of its proximal end, in the finger with the exception of the last phalanx.

28. The method according to claim 24, wherein the composition is in the form of an implant optionally having a sharpened end, a cylindrical tube or a thread and the composition is administered subcutaneously at a distance greater than 1 cm from the edge of the nail on the side of its proximal end, with the exception of the last phalanx.

29. The method according to claim 20, wherein the composition is a biocompatible polymer with prolonged release, biodegradable or bio-resorbable, preferably a polyester, and even more preferably a lactic acid-glycolic acid copolymer (PLGA) or a polymer of lactic acid (PLA), and wherein the active substance content is between 0 and 99%, preferably less than or equal to 85% and even more preferably between 20 and 70% by weight and wherein the composition comprises between 0.04 and 6.5 mg of ciclopirox, ciclopiroxolamine, terbinafine or terbinafine hydrochloride, or itraconazole.

30. The method according to claim 24, wherein the composition is a biocompatible polymer with prolonged release, biodegradable or bio-resorbable, preferably a polyester, and even more preferably a lactic acid-glycolic acid copolymer (PLGA) or a polymer of lactic acid (PLA), and wherein the active substance content is between 0 and 99%, preferably less than or equal to 85% and even more preferably between 20 and 70% by weight and wherein the composition comprises between 0.04 and 6.5 mg of ciclopirox, ciclopiroxolamine, terbinafine or terbinafine hydrochloride, or itraconazole.

31. The method according to claim 20, wherein the method is for the treatment of nail mycoses, wherein the composition is in the form of an implant comprising one or more solid biodegradable sustained-release polymers and optionally one or more antifungal agents and the composition is in the form of an implant having a maximum length of 25 mm, preferably between 4 and 15 mm, and a diameter of between 0 and 0.1 and 1 mm preferably between 0.3 and 0.8 mm, implant intended to be administered subcutaneously at a distance of more than 1 cm from the edge of the nail, preferably in the finger except for the last phalanx.

32. The method according to claim 24, wherein the method comprises administering a composition in the form of an implant, comprising one or more solid biodegradable sustained-release polymers, and one or more active substances selected from:
cyclosporins;
prostaglandins;
Echisetum arvense (horsetail), Chitosan, Hydroxypropyl chitosan, Hyaluronic acid, Arginine, Biotin, Valproic acid, Growth hormone (GH), Minoxidil and/or Finasteride;
Vasodilators;
valproic acid;
and possibly one or more active substances selected from:
anti-infectives selected from:
antifungal agents,
Antibiotics,
and pest control products;
anti-inflammatory drugs selected from
steroidal anti-inflammatory drugs,
non-steroidal anti-inflammatory drugs, and
Janus Kinase inhibitors (JAK);
vitamin D or vitamin D derivatives;
immunosuppressants;
amino acids;
active substances promoting blood circulation chosen from among vasodilators, and
anti-platelet aggregants;
local anaesthetics,
and the composition is in the form of an implant having a maximum length of 25 mm preferably between 4 and 15 mm and a diameter of between 0.1 and 1 mm preferably between 0.3 and 0.8 mm, the implant being intended to be administered subcutaneously at a distance of more than 1 cm from the edge of the nail on the side of its proximal end preferably in the finger with the exception of the last phalanx.

33. The method according to claim 20, wherein the method comprises administering a composition that is in the form of an implant, a cylindrical tube or a thread, optionally having a sharpened end, and wherein the composition is administered in the finger, with the exception of the last phalanx, in the first or second phalanx of the fingers, preferably the second phalanx, and in the first phalanx of the thumb or big toe, wherein one or more compositions, preferably from one to eight compositions, more preferably from one to six compositions, even more preferably from one to four compositions are administered simultaneously in the same finger and wherein the treatment is continuous, preferably for a minimum of 48 weeks when the whole nail is affected, a minimum of 24 weeks when less than half of the nail is affected, a minimum of 12 weeks when less than a quarter of the nail is affected, or a maximum of 6 weeks when less than 10% is affected.

34. The method according to claim 20, wherein the method comprises administering a solid composition comprising one or more solid polymers, biodegradable or not, with sustained release, and optionally one or more active substances and the composition is in the form of a device which is administered by insertion into the space between the bed and the nail plate and wherein the composition is optionally used simultaneously or spread out over time with subcutaneous administration at a distance greater than 1 cm from the edge of the nail on the side of its proximal end in the finger, with the exception of the last phalanx of one or more compositions comprising one or more solid biodegradable sustained-release polymers, and optionally one or more active substances.

35. The method according to claim 20, wherein the method comprises administering a liquid or non-solid composition comprising one or more biodegradable or non-biodegradable polymers with prolonged release, and optionally one or more active substances, preferably one or more antifungal agents, wherein the composition is in the form of a device intended to be administered by insertion into the space between the bed and the nail plate and wherein the composition is optionally used simultaneously or spread out in time with subcutaneous administration at a distance greater than 1 cm from the edge of the nail on the side of its proximal end in the finger, with the exception of the last phalanx of one or more compositions comprising one or more solid biodegradable sustained-release polymers, and optionally one or more active substances.

36. The method according to claim 20, wherein the method comprises administering a nail varnish with antimycotic action comprising one or more antifungals selected from fluconazole, ketoconazole, miconazole, tioconazole, tavaborole, terbinafine, terbinafine hydrochloride, tolnaftate, amorolfine, ciclopirox, ciclopiroxolamine, terbinafine, terbinafine hydrochloride or miconazole, for its use in the treatment of nail diseases by application to the nails simultaneously or staggered in time with the administration in the finger subcutaneously at a distance of more than 1 cm from the edge of the nail on the side of its proximal end with the exception of the last phalanx of one or more compositions comprising one or more solid biodegradable sustained-release polymers, and optionally one or more active substances and/or with the administration by insertion into the space between the bed and the nail plate of one or more solid compositions, liquid or non-solid comprising one or more solid biodegradable sustained-release polymers, and optionally one or more active substances.

37. The method according to claim 20, wherein the method comprises administering an antifungal pharmaceutical composition comprising one or more antifungals selected from azole derivatives such as ketoconazole, fluconazole, isavuconazole, itraconazole, posaconazole or voriconazole; echinocandins such as anidulafungin, caspofungin, micafungin, flucytosine, griseofulvin, terbinafine hydrochloride and terbinafine; amphotericin B for its use in the treatment of nail diseases by oral or injectable administration simultaneously or over time with the administration by the sub-therapeutic route; amphotericin B for its use in the treatment of nail diseases by oral or injectable administration simultaneously or over time with the administration by the sub-therapeutic route. cutaneous in the finger at a distance greater than 1 cm from the edge of the nail on the side of its proximal end with the exception of the last phalanx of one or more compositions comprising one or more solid biodegradable sustained-release polymers, and optionally one or more active substances and/or with the administration by insertion into the space between the bed and the nail plate of one or more solid compositions, liquid or non-solid comprising one or more solid biodegradable sustained-release polymers, and optionally one or more active substances and/or with the application to the nails of a nail varnish with antimycotic action comprising one or more antifungals.

38. The method according to claim 20, wherein the method comprises administering a composition comprising one or more solid biodegradable sustained-release polymers, and optionally one or more active substances, wherein the composition is in the form of an implant, optionally with a sharpened end, a cylindrical tube or a thread, and wherein the composition is administered in the finger at a distance of more than 1 cm from the edge of the nail at its proximal end, with the exception of the last phalanx, for use in the treatment of nail diseases by simultaneous or staggered administration with:
the administration by insertion into the space between the bed and the nail plate of a solid composition comprising one or more biodegradable or non-biodegradable sustained-release polymers, and optionally one or more active substances, preferably one or more antifungal agents
and/or
the administration by insertion into the space between the bed and the nail plate of a non-solid composition comprising one or more biodegradable or non-biodegradable sustained-release polymers, and optionally one or more active substances, preferably one or more antifungal agents.
and/or
the application to the nails of a nail varnish with antimycotic action according to claim 17 comprising one or more antifungals selected from fluconazole, ketoconazole, miconazole, tioconazole, tavaborole, terbinafine, terbinafine hydrochloride, ciclopiroxolamine, tolnaftate, amorolfine, ciclopirox, tioconazole or miconazole,
and/or
the oral or injectable administration of an antifungal pharmaceutical composition comprising one or more antifungals selected from azole derivatives such as ketoconazole, fluconazole, isavuconazole, traconazole, posaconazole or voriconazole; echinocandins such as anidulafungin, caspofungin, micafungin, flucytosine, griseofulvin, terbinafine hydrochloride and terbinafine; amphotericin B
and/or
a laser treatment causing the destruction of the fungus by photobiological thermal effect.
